SEQUENCE LISTING (178 sequences)

SEQ ID NO: 1 - DNA, 21 bp, Artificial Sequence
  Oligo
ttctaatacgactcactatag

SEQ ID NO: 2 - DNA, 14 bp, Artificial Sequence
  Oligo
gttttagagctaga

SEQ ID NO: 3 - DNA, 79 bp, Artificial Sequence
  Oligo
aaaagcaccgactcggtgccactttttaagttgataacggactagccttattttaacttgctatttctagctctaaaac

SEQ ID NO: 4 - DNA, 20 bp, Artificial Sequence
  Oligo
gcagtttctgccgtgcttaa

SEQ ID NO: 5 - DNA, 20 bp, Artificial Sequence
  Oligo
cggaacagcgcccagccttt

SEQ ID NO: 6 - DNA, 20 bp, Artificial Sequence
  Oligo
ttcggtcccttctgtaagaa

SEQ ID NO: 7 - DNA, 20 bp, Artificial Sequence
  Oligo
cagaaacgactccagtaccg

SEQ ID NO: 8 - DNA, 20 bp, Artificial Sequence
  Oligo
ctgtagctgctgaaacgttg

SEQ ID NO: 9 - DNA, 20 bp, Artificial Sequence
  Oligo
acaggtatcgtttggaggca

SEQ ID NO: 10 - DNA, 20 bp, Artificial Sequence
  Oligo
agttacccctctaagtaatg

SEQ ID NO: 11 - DNA, 20 bp, Artificial Sequence
  Oligo
ccatgcaacatgaataacag

SEQ ID NO: 12 - DNA, 20 bp, Artificial Sequence
  Oligo
tttcctctgtcattacgtca

SEQ ID NO: 13 - DNA, 20 bp, Artificial Sequence
  Oligo
cgactattgataaaaatcaa

SEQ ID NO: 14 - DNA, 20 bp, Artificial Sequence
  Oligo
atgttttcacttaatagtat

SEQ ID NO: 15 - DNA, 20 bp, Artificial Sequence
  Oligo
tgcgcttgctcttcatctag

SEQ ID NO: 16 - DNA, 55 bp, Artificial Sequence
  Oligo
ttctaatacgactcactataggcagtttctgccgtgcttaagttttagagctaga

SEQ ID NO: 17 - DNA, 55 bp, Artificial Sequence
  Oligo
ttctaatacgactcactatagcggaacagcgcccagcctttgttttagagctaga

SEQ ID NO: 18 - DNA, 55 bp, Artificial Sequence
  Oligo
ttctaatacgactcactatagttcggtcccttctgtaagaagttttagagctaga

SEQ ID NO: 19 - DNA, 55 bp, Artificial Sequence
  Oligo
ttctaatacgactcactatagcagaaacgactccagtaccggttttagagctaga

SEQ ID NO: 20 - DNA, 55 bp, Artificial Sequence
  Oligo
ttctaatacgactcactatagctgtagctgctgaaacgttggttttagagctaga

SEQ ID NO: 21 - DNA, 55 bp, Artificial Sequence
  Oligo
ttctaatacgactcactatagacaggtatcgtttggaggcagttttagagctaga

SEQ ID NO: 22 - DNA, 55 bp, Artificial Sequence
  Oligo
ttctaatacgactcactatagagttacccctctaagtaatggttttagagctaga

SEQ ID NO: 23 - DNA, 55 bp, Artificial Sequence
  Oligo
ttctaatacgactcactatagccatgcaacatgaataacaggttttagagctaga

SEQ ID NO: 24 - DNA, 55 bp, Artificial Sequence
  Oligo
ttctaatacgactcactatagtttcctctgtcattacgtcagttttagagctaga

SEQ ID NO: 25 - DNA, 55 bp, Artificial Sequence
  Oligo
ttctaatacgactcactatagcgactattgataaaaatcaagttttagagctaga

SEQ ID NO: 26 - DNA, 55 bp, Artificial Sequence
  Oligo
ttctaatacgactcactatagatgttttcacttaatagtatgttttagagctaga

SEQ ID NO: 27 - DNA, 55 bp, Artificial Sequence
  Oligo
ttctaatacgactcactatagtgcgcttgctcttcatctaggttttagagctaga

SEQ ID NO: 28 - DNA, 20 bp, Artificial Sequence
  Oligo
ccagccagcacagaaacatc

SEQ ID NO: 29 - DNA, 20 bp, Artificial Sequence
  Oligo
agcggcagccataaggtgga

SEQ ID NO: 30 - DNA, 20 bp, Artificial Sequence
  Oligo
aggtcttcatcgtccacctc

SEQ ID NO: 31 - DNA, 20 bp, Artificial Sequence
  Oligo
ttcggtcccttctgtaagaa

SEQ ID NO: 32 - DNA, 20 bp, Artificial Sequence
  Oligo
tgaatgacttccccaattat

SEQ ID NO: 33 - DNA, 20 bp, Artificial Sequence
  Oligo
ctgtagctgctgaaacgttg

SEQ ID NO: 34 - DNA, 20 bp, Artificial Sequence
  Oligo
tgatttaactataccttttg

SEQ ID NO: 35 - DNA, 20 bp, Artificial Sequence
  Oligo
cgccgaacgattagctcttc

SEQ ID NO: 36 - DNA, 20 bp, Artificial Sequence
  Oligo
cgactattgataaaaatcaa

SEQ ID NO: 37 - DNA, 20 bp, Artificial Sequence
  Oligo
cagtttgatgagtatagaaa

SEQ ID NO: 38 - DNA, 20 bp, Artificial Sequence
  Oligo
gaaggttttaccaatggctc

SEQ ID NO: 39 - DNA, 20 bp, Artificial Sequence
  Oligo
atgttttcacttaatagtat

SEQ ID NO: 40 - DNA, 55 bp, Artificial Sequence
  Oligo
ttctaatacgactcactatagccagccagcacagaaacatcgttttagagctaga

SEQ ID NO: 41 - DNA, 55 bp, Artificial Sequence
  Oligo
ttctaatacgactcactatagagcggcagccataaggtggagttttagagctaga

SEQ ID NO: 42 - DNA, 55 bp, Artificial Sequence
  Oligo
ttctaatacgactcactatagaggtcttcatcgtccacctcgttttagagctaga

SEQ ID NO: 43 - DNA, 55 bp, Artificial Sequence; identical to SEQ ID NO: 18
  Oligo

SEQ ID NO: 44 - DNA, 55 bp, Artificial Sequence
  Oligo
ttctaatacgactcactatagtgaatgacttccccaattatgttttagagctaga

SEQ ID NO: 45 - DNA, 55 bp, Artificial Sequence; identical to SEQ ID NO: 20
  Oligo

SEQ ID NO: 46 - DNA, 55 bp, Artificial Sequence
  Oligo
ttctaatacgactcactatagtgatttaactataccttttggttttagagctaga

SEQ ID NO: 47 - DNA, 55 bp, Artificial Sequence
  Oligo
ttctaatacgactcactatagcgccgaacgattagctcttcgttttagagctaga

SEQ ID NO: 48 - DNA, 55 bp, Artificial Sequence; identical to SEQ ID NO: 25
  Oligo

SEQ ID NO: 49 - DNA, 55 bp, Artificial Sequence
  Oligo
ttctaatacgactcactatagcagtttgatgagtatagaaagttttagagctaga

SEQ ID NO: 50 - DNA, 55 bp, Artificial Sequence
  Oligo
ttctaatacgactcactataggaaggttttaccaatggctcgttttagagctaga

SEQ ID NO: 51 - DNA, 55 bp, Artificial Sequence; identical to SEQ ID NO: 26
  Oligo

SEQ ID NO: 52 - DNA, 20 bp, Artificial Sequence
  Oligo
tatgcaccgccagtataagt

SEQ ID NO: 53 - DNA, 20 bp, Artificial Sequence
  Oligo
aaaaataatgttgcatcaat

SEQ ID NO: 54 - DNA, 20 bp, Artificial Sequence
  Oligo
gtccttctcgttaaaaaatc

SEQ ID NO: 55 - DNA, 20 bp, Artificial Sequence
  Oligo
tgctatcaatgattcccgct

SEQ ID NO: 56 - DNA, 20 bp, Artificial Sequence
  Oligo
gaaaaacctgatgtttacat

SEQ ID NO: 57 - DNA, 20 bp, Artificial Sequence
  Oligo
tccgcaatttgctcaatttc

SEQ ID NO: 58 - DNA, 20 bp, Artificial Sequence
  Oligo
tcgtcatgctcaatggcgtt

SEQ ID NO: 59 - DNA, 20 bp, Artificial Sequence
  Oligo
aagaccaaatttcaaagtca

SEQ ID NO: 60 - DNA, 20 bp, Artificial Sequence
  Oligo
gactggggattattcgcagg

SEQ ID NO: 61 - DNA, 20 bp, Artificial Sequence
  Oligo
aacttggttaccatcccaat

SEQ ID NO: 62 - DNA, 20 bp, Artificial Sequence
  Oligo
aatgatgttgaattccaagt

SEQ ID NO: 63 - DNA, 20 bp, Artificial Sequence
  Oligo
tgcattgcgaggattagcaa

SEQ ID NO: 64 - DNA, 20 bp, Artificial Sequence
  Oligo
aagaataaaagtggccaaat

SEQ ID NO: 65 - DNA, 20 bp, Artificial Sequence
  Oligo
gctgtgccgttgtttgtatt

SEQ ID NO: 66 - DNA, 20 bp, Artificial Sequence
  Oligo
caatttttagatcgcttacg

SEQ ID NO: 67 - DNA, 20 bp, Artificial Sequence
  Oligo
tgcgtaataattgtccgctt

SEQ ID NO: 68 - DNA, 20 bp, Artificial Sequence
  Oligo
ggcattcaagatattatcac

SEQ ID NO: 69 - DNA, 20 bp, Artificial Sequence
  Oligo
taggaggtttgcgaactacg

SEQ ID NO: 70 - DNA, 20 bp, Artificial Sequence
  Oligo
cccgtatcctttggtgcggt

SEQ ID NO: 71 - DNA, 20 bp, Artificial Sequence
  Oligo
caaggtaaggcaacataaga

SEQ ID NO: 72 - DNA, 20 bp, Artificial Sequence
  Oligo
ccaaacgtaacttgcttaat

SEQ ID NO: 73 - DNA, 20 bp, Artificial Sequence
  Oligo
cataatttccgccttttatt

SEQ ID NO: 74 - DNA, 20 bp, Artificial Sequence
  Oligo
gatgatatgattgatactgg

SEQ ID NO: 75 - DNA, 20 bp, Artificial Sequence
  Oligo
tggcgagcatagccgaaata

SEQ ID NO: 76 - DNA, 20 bp, Artificial Sequence
  Oligo
tataaaattattgaatgggt

SEQ ID NO: 77 - DNA, 20 bp, Artificial Sequence
  Oligo
ataggtaagaataaaccacg

SEQ ID NO: 78 - DNA, 20 bp, Artificial Sequence
  Oligo
catgatgaaccgtgagagag

SEQ ID NO: 79 - DNA, 20 bp, Artificial Sequence
  Oligo
tcaaacagttaatttgagta

SEQ ID NO: 80 - DNA, 20 bp, Artificial Sequence
  Oligo
gcgataattaaaactaaaat

SEQ ID NO: 81 - DNA, 20 bp, Artificial Sequence
  Oligo
gtgggaattaaatcaatgtc

SEQ ID NO: 82 - DNA, 20 bp, Artificial Sequence
  Oligo
cttgaaaaaattatcgcagc

SEQ ID NO: 83 - DNA, 20 bp, Artificial Sequence
  Oligo
gagcaccaccttgacatggt

SEQ ID NO: 84 - DNA, 20 bp, Artificial Sequence
  Oligo
gagaattaatacgatagcct

SEQ ID NO: 85 - DNA, 20 bp, Artificial Sequence
  Oligo
ggtcgccgtcaaatcgattt

SEQ ID NO: 86 - DNA, 20 bp, Artificial Sequence
  Oligo
actctcattagagacgtttt

SEQ ID NO: 87 - DNA, 20 bp, Artificial Sequence
  Oligo
cctgccggtcgcaagattgt

SEQ ID NO: 88 - DNA, 20 bp, Artificial Sequence
  Oligo
ttttgtgcctgcgtatttgt

SEQ ID NO: 89 - DNA, 20 bp, Artificial Sequence
  Oligo
tgattttatcaatggcaagg

SEQ ID NO: 90 - DNA, 20 bp, Artificial Sequence
  Oligo
ttccggcgtatccgcccaag

SEQ ID NO: 91 - DNA, 20 bp, Artificial Sequence
  Oligo
tggaggtgctcaagttatgt

SEQ ID NO: 92 - DNA, 20 bp, Artificial Sequence
  Oligo
ataaacacttccccactact

SEQ ID NO: 93 - DNA, 20 bp, Artificial Sequence
  Oligo
tggtggggaacgtcagcgtg

SEQ ID NO: 94 - DNA, 20 bp, Artificial Sequence
  Oligo
attgatgaaaaaccaattgg

SEQ ID NO: 95 - DNA, 20 bp, Artificial Sequence
  Oligo
gtttttattcgtgtaatata

SEQ ID NO: 96 - DNA, 20 bp, Artificial Sequence
  Oligo
gaggtttaatatgtctaaag

SEQ ID NO: 97 - DNA, 20 bp, Artificial Sequence
  Oligo
ttaggtacagttatccgtgg

SEQ ID NO: 98 - DNA, 20 bp, Artificial Sequence
  Oligo
ttttttcttttgttctttag

SEQ ID NO: 99 - DNA, 20 bp, Artificial Sequence
  Oligo
gttgttttaaacgaaaaatg

SEQ ID NO: 100 - DNA, 20 bp, Artificial Sequence
  Oligo
aatttagtgcctgcatttaa

SEQ ID NO: 101 - DNA, 20 bp, Artificial Sequence
  Oligo
ttgataagaatcgccaatat

SEQ ID NO: 102 - DNA, 20 bp, Artificial Sequence
  Oligo
catatttctgtaaaatattg

SEQ ID NO: 103 - DNA, 20 bp, Artificial Sequence
  Oligo
gcagaacgttatatcggcgg

SEQ ID NO: 104 - DNA, 20 bp, Artificial Sequence
  Oligo
gggcgcaaaattcaatcagg

SEQ ID NO: 105 - DNA, 20 bp, Artificial Sequence
  Oligo
gtcggttcgagtccgaccct

SEQ ID NO: 106 - DNA, 20 bp, Artificial Sequence
  Oligo
aattggccgcactcacttaa

SEQ ID NO: 107 - DNA, 20 bp, Artificial Sequence
  Oligo
aatttcatgtggcattgatg

SEQ ID NO: 108 - DNA, 55 bp, Artificial Sequence
  Oligo
ttctaatacgactcactatagtatgcaccgccagtataagtgttttagagctaga

SEQ ID NO: 109 - DNA, 55 bp, Artificial Sequence
  Oligo
ttctaatacgactcactatagaaaaataatgttgcatcaatgttttagagctaga

SEQ ID NO: 110 - DNA, 55 bp, Artificial Sequence
  Oligo
ttctaatacgactcactataggtccttctcgttaaaaaatcgttttagagctaga

SEQ ID NO: 111 - DNA, 55 bp, Artificial Sequence
  Oligo
ttctaatacgactcactatagtgctatcaatgattcccgctgttttagagctaga

SEQ ID NO: 112 - DNA, 55 bp, Artificial Sequence
  Oligo
ttctaatacgactcactataggaaaaacctgatgtttacatgttttagagctaga

SEQ ID NO: 113 - DNA, 55 bp, Artificial Sequence
  Oligo
ttctaatacgactcactatagtccgcaatttgctcaatttcgttttagagctaga

SEQ ID NO: 114 - DNA, 55 bp, Artificial Sequence
  Oligo
ttctaatacgactcactatagtcgtcatgctcaatggcgttgttttagagctaga

SEQ ID NO: 115 - DNA, 55 bp, Artificial Sequence
  Oligo
ttctaatacgactcactatagaagaccaaatttcaaagtcagttttagagctaga

SEQ ID NO: 116 - DNA, 55 bp, Artificial Sequence
  Oligo
ttctaatacgactcactataggactggggattattcgcagggttttagagctaga

SEQ ID NO: 117 - DNA, 55 bp, Artificial Sequence
  Oligo
ttctaatacgactcactatagaacttggttaccatcccaatgttttagagctaga

SEQ ID NO: 118 - DNA, 55 bp, Artificial Sequence
  Oligo
ttctaatacgactcactatagaatgatgttgaattccaagtgttttagagctaga

SEQ ID NO: 119 - DNA, 55 bp, Artificial Sequence
  Oligo
ttctaatacgactcactatagtgcattgcgaggattagcaagttttagagctaga

SEQ ID NO: 120 - DNA, 55 bp, Artificial Sequence
  Oligo
ttctaatacgactcactatagaagaataaaagtggccaaatgttttagagctaga

SEQ ID NO: 121 - DNA, 55 bp, Artificial Sequence
  Oligo
ttctaatacgactcactataggctgtgccgttgtttgtattgttttagagctaga

SEQ ID NO: 122 - DNA, 55 bp, Artificial Sequence
  Oligo
ttctaatacgactcactatagcaatttttagatcgcttacggttttagagctaga

SEQ ID NO: 123 - DNA, 55 bp, Artificial Sequence
  Oligo
ttctaatacgactcactatagtgcgtaataattgtccgcttgttttagagctaga

SEQ ID NO: 124 - DNA, 55 bp, Artificial Sequence
  Oligo
ttctaatacgactcactatagggcattcaagatattatcacgttttagagctaga

SEQ ID NO: 125 - DNA, 55 bp, Artificial Sequence
  Oligo
ttctaatacgactcactatagtaggaggtttgcgaactacggttttagagctaga

SEQ ID NO: 126 - DNA, 55 bp, Artificial Sequence
  Oligo
ttctaatacgactcactatagcccgtatcctttggtgcggtgttttagagctaga

SEQ ID NO: 127 - DNA, 55 bp, Artificial Sequence
  Oligo
ttctaatacgactcactatagcaaggtaaggcaacataagagttttagagctaga

SEQ ID NO: 128 - DNA, 55 bp, Artificial Sequence
  Oligo
ttctaatacgactcactatagccaaacgtaacttgcttaatgttttagagctaga

SEQ ID NO: 129 - DNA, 55 bp, Artificial Sequence
  Oligo
ttctaatacgactcactatagcataatttccgccttttattgttttagagctaga

SEQ ID NO: 130 - DNA, 55 bp, Artificial Sequence
  Oligo
ttctaatacgactcactataggatgatatgattgatactgggttttagagctaga

SEQ ID NO: 131 - DNA, 55 bp, Artificial Sequence
  Oligo
ttctaatacgactcactatagtggcgagcatagccgaaatagttttagagctaga

SEQ ID NO: 132 - DNA, 55 bp, Artificial Sequence
  Oligo
ttctaatacgactcactatagtataaaattattgaatgggtgttttagagctaga

SEQ ID NO: 133 - DNA, 55 bp, Artificial Sequence
  Oligo
ttctaatacgactcactatagataggtaagaataaaccacggttttagagctaga

SEQ ID NO: 134 - DNA, 55 bp, Artificial Sequence
  Oligo
ttctaatacgactcactatagcatgatgaaccgtgagagaggttttagagctaga

SEQ ID NO: 135 - DNA, 55 bp, Artificial Sequence
  Oligo
ttctaatacgactcactatagtcaaacagttaatttgagtagttttagagctaga

SEQ ID NO: 136 - DNA, 55 bp, Artificial Sequence
  Oligo
ttctaatacgactcactataggcgataattaaaactaaaatgttttagagctaga

SEQ ID NO: 137 - DNA, 55 bp, Artificial Sequence
  Oligo
ttctaatacgactcactataggtgggaattaaatcaatgtcgttttagagctaga

SEQ ID NO: 138 - DNA, 55 bp, Artificial Sequence
  Oligo
ttctaatacgactcactatagcttgaaaaaattatcgcagcgttttagagctaga

SEQ ID NO: 139 - DNA, 55 bp, Artificial Sequence
  Oligo
ttctaatacgactcactataggagcaccaccttgacatggtgttttagagctaga

SEQ ID NO: 140 - DNA, 55 bp, Artificial Sequence
  Oligo
ttctaatacgactcactataggagaattaatacgatagcctgttttagagctaga

SEQ ID NO: 141 - DNA, 55 bp, Artificial Sequence
  Oligo
ttctaatacgactcactatagggtcgccgtcaaatcgatttgttttagagctaga

SEQ ID NO: 142 - DNA, 55 bp, Artificial Sequence
  Oligo
ttctaatacgactcactatagactctcattagagacgttttgttttagagctaga

SEQ ID NO: 143 - DNA, 55 bp, Artificial Sequence
  Oligo
ttctaatacgactcactatagcctgccggtcgcaagattgtgttttagagctaga

SEQ ID NO: 144 - DNA, 55 bp, Artificial Sequence
  Oligo
ttctaatacgactcactatagttttgtgcctgcgtatttgtgttttagagctaga

SEQ ID NO: 145 - DNA, 55 bp, Artificial Sequence
  Oligo
ttctaatacgactcactatagtgattttatcaatggcaagggttttagagctaga

SEQ ID NO: 146 - DNA, 55 bp, Artificial Sequence
  Oligo
ttctaatacgactcactatagttccggcgtatccgcccaaggttttagagctaga

SEQ ID NO: 147 - DNA, 55 bp, Artificial Sequence
  Oligo
ttctaatacgactcactatagtggaggtgctcaagttatgtgttttagagctaga

SEQ ID NO: 148 - DNA, 55 bp, Artificial Sequence
  Oligo
ttctaatacgactcactatagataaacacttccccactactgttttagagctaga

SEQ ID NO: 149 - DNA, 55 bp, Artificial Sequence
  Oligo
ttctaatacgactcactatagtggtggggaacgtcagcgtggttttagagctaga

SEQ ID NO: 150 - DNA, 55 bp, Artificial Sequence
  Oligo
ttctaatacgactcactatagattgatgaaaaaccaattgggttttagagctaga

SEQ ID NO: 151 - DNA, 55 bp, Artificial Sequence
  Oligo
ttctaatacgactcactataggtttttattcgtgtaatatagttttagagctaga

SEQ ID NO: 152 - DNA, 55 bp, Artificial Sequence
  Oligo
ttctaatacgactcactataggaggtttaatatgtctaaaggttttagagctaga

SEQ ID NO: 153 - DNA, 55 bp, Artificial Sequence
  Oligo
ttctaatacgactcactatagttaggtacagttatccgtgggttttagagctaga

SEQ ID NO: 154 - DNA, 55 bp, Artificial Sequence
  Oligo
ttctaatacgactcactatagttttttcttttgttctttaggttttagagctaga

SEQ ID NO: 155 - DNA, 55 bp, Artificial Sequence
  Oligo
ttctaatacgactcactataggttgttttaaacgaaaaatggttttagagctaga

SEQ ID NO: 156 - DNA, 55 bp, Artificial Sequence
  Oligo
ttctaatacgactcactatagaatttagtgcctgcatttaagttttagagctaga

SEQ ID NO: 157 - DNA, 55 bp, Artificial Sequence
  Oligo
ttctaatacgactcactatagttgataagaatcgccaatatgttttagagctaga

SEQ ID NO: 158 - DNA, 55 bp, Artificial Sequence
  Oligo
ttctaatacgactcactatagcatatttctgtaaaatattggttttagagctaga

SEQ ID NO: 159 - DNA, 55 bp, Artificial Sequence
  Oligo
ttctaatacgactcactataggcagaacgttatatcggcgggttttagagctaga

SEQ ID NO: 160 - DNA, 55 bp, Artificial Sequence
  Oligo
ttctaatacgactcactataggggcgcaaaattcaatcagggttttagagctaga

SEQ ID NO: 161 - DNA, 55 bp, Artificial Sequence
  Oligo
ttctaatacgactcactataggtcggttcgagtccgaccctgttttagagctaga

SEQ ID NO: 162 - DNA, 55 bp, Artificial Sequence
  Oligo
ttctaatacgactcactatagaattggccgcactcacttaagttttagagctaga

SEQ ID NO: 163 - DNA, 55 bp, Artificial Sequence
  Oligo
ttctaatacgactcactatagaatttcatgtggcattgatggttttagagctaga

SEQ ID NO: 164 - protein, 6 aa, Artificial Sequence
  PAM
NNGRRT

SEQ ID NO: 165 - protein, 4 aa, Artificial Sequence
  PAM
TTTV

SEQ ID NO: 166 - protein, 4 aa, Artificial Sequence
  PAM
TYCV

SEQ ID NO: 167 - protein, 4 aa, Artificial Sequence
  PAM
TYCV

SEQ ID NO: 168 - protein, 4 aa, Artificial Sequence
  PAM
TATV

SEQ ID NO: 169 - protein, 8 aa, Artificial Sequence
  PAM
NNNNRYAC

SEQ ID NO: 170 - protein, 8 aa, Artificial Sequence
  PAM
NNNNGATT

SEQ ID NO: 171 - protein, 7 aa, Artificial Sequence
  PAM
NNAGAAW

SEQ ID NO: 172 - protein, 6 aa, Artificial Sequence
  PAM
NAAAAC

SEQ ID NO: 173 - DNA, 21 bp, Artificial Sequence
  Oligo
gagaatctgcaagtggatatt

SEQ ID NO: 174 - protein, 4 aa, Artificial Sequence
  PAM
NGCG

SEQ ID NO: 175 - protein, 4 aa, Artificial Sequence
  PAM
NGAG

SEQ ID NO: 176 - protein, 4 aa, Artificial Sequence
  PAM
NGAN

SEQ ID NO: 177 - protein, 4 aa, Artificial Sequence
  PAM
NGNG

SEQ ID NO: 178 - protein, 6 aa, Artificial Sequence
  PAM
NNGRRN

(19) United States
(12) Patent Application Publication
(10) Pub. No.: US 2024/0035024 A1
(43) Pub. Date: Feb. 1, 2024

(54) LINKED-READ SEQUENCING LIBRARY PREPARATION

(71) Applicant: Drexel University, Philadelphia, PA (US)

(72) Inventors: Ming Xiao, Huntingdon Valley, PA (US); Lahari Uppuluri, Philadelphia, PA (US)

(21) Appl. No.: 18/031,717

(22) PCT Filed: Oct. 15, 2021
(86) PCT No.: PCT/US21/55118

§ 371 Date: Apr. 13, 2023

Related U.S. Application Data
(60) Provisional application No. 63/092,973, filed on Oct. 16, 2020.

(51) Int. Cl. C12N 15/10; C12Q 1/6806; C12Q 1/6874

(52) U.S. Cl. CPC C12N 15/1093; C12Q 1/6806; C12Q 1/6874; C12N 2310/20

(57) ABSTRACT

The present invention relates to innovative means of generating sequence-linked DNA fragments and subsequent uses of such linked DNA fragments for de novo haplotype-resolved whole genome mapping and massively parallel sequencing. In various embodiments described herein, the methods of the invention relate to methods of generating linked-paired end nucleic acid fragments sharing common linker nucleic acid sequences using a computationally-designed sgRNA library together with a nicking RNA-guided endonuclease, methods of analyzing the nucleotides sequences from the linked-paired-end sequenced fragments, and methods of de novo whole genome mapping. Thus, the methods of this invention allow establishing sequence contiguity across the whole genome, and achieving high-quality, low-cost de novo assembly of complex genomes.

### CROSS-REFERENCE TO RELATED APPLICATIONS

This application claims priority under 35 U.S.C. § 119(e) to U.S. Provisional Patent Application No. 63/092,973, filed Oct. 16, 2020, the disclosures of which is incorporated herein by reference in its entirety.

### SEQUENCE LISTING

The ASCII text file named “046528-7110WO1_Sequence listing ST25” created on Oct. 7, 2021, comprising 31 Kbytes, is hereby incorporated by reference in its entirety.

### BACKGROUND OF THE INVENTION

Genomics holds much promise for huge improvements in human healthcare. Despite major advances in high-throughput sequencing, genomics faces several practical challenges. Accurate de novo genome assembly of sequence reads and structural variant analysis using “short read” shotgun sequencing remains challenging and represent the weak link in genome projects. Most re-sequencing projects rely on mapping the sequencing data to the reference sequence to identify variants of interest. When whole genome assembly is attempted, it is done by paired-end sequencing of cloned genomic DNA fragments to provide scaffolds for assembly. Cloning of large DNA fragments is difficult. Therefore, small insert libraries of varying sizes have been prepared for paired-end sequencing, thus limiting the resolution of haplotypes and increasing the complexity, time, and cost of the sequencing project. In addition, complex genomic loci, such as the major histocompatibility (MHC) region, are important for infectious and autoimmune diseases. These regions contain highly repetitive sequences and are particularly challenging for sequence assembly. As such, robust technologies that can aid in de novo sequence assembly are sorely needed as whole genome sequencing becomes more widely adopted.

Emerging whole genome scanning techniques reveal the prevalence and importance of structural variation including copy-number variations, deletions, insertions, inversions and translocations. Detecting copy number variation often relies on detection of relative signal intensities by array-based or quantitative PCR-based technologies. Array-based methods, such as array-based comparative genomic hybridization (aCGH), have been used extensively in interrogation of copy number variation in the human genome. Except for deletions, however, these methods do not provide positional information regarding the locations of copy number variants (CNVs) and cannot detect balanced structural variation, such as inversions or translocations. Paired-end mapping techniques, traditionally by Sanger sequencing and now by next-generation sequencing, generally have low sensitivity in repetitive regions, where most of the structural variation lies. Recent efforts to characterize CNVs in human genomes at high resolution involve paired-end mapping of clones, but this approach, while useful for exploratory studies in this small sample set, is too labor-intensive and time-consuming to be applicable for analysis of large numbers of individuals. Furthermore, the resolution is no better than 8 kb.

Restriction mapping was instrumental in the Human Genome Project. One approach to address drawbacks of traditional restriction mapping is optical mapping. In this approach, large DNA fragments are stretched and immobilized on glass slides and cut in situ with restriction enzymes. Optical mapping was used to construct ordered restriction maps for whole genomes, and it provided scaffolds for shotgun sequence assembly and validation. This method, however, is limited by its low throughput, non-uniform DNA stretching, imprecise DNA length measurement, and high error rates.

Therefore, despite all developments in high throughput sequencing, there remains a need in the art for novel methods of sequencing whole genomes with great accuracy, low cost and within a reasonable timeline. This disclosure addresses that need.

### SUMMARY OF THE INVENTION

According to a first aspect of the invention, a method of preparing a DNA sequencing library comprising DNA fragments having linked-paired ends from at least one double-stranded DNA sample having a first and a second DNA strand is provided, the method comprises: (a) obtaining a single guide RNA (sgRNA) library comprising multiple sgRNA pairs, wherein: (i) each sgRNA pair comprises a first sgRNA and a second sgRNA, and (ii) the first sgRNA of each sgRNA pair targets a first target DNA sequence on the first DNA strand and the second sgRNA of each sgRNA pair targets a second target DNA sequence on the second DNA strand; (b) contacting the double-stranded DNA sample with the sgRNA library and at least one nickase, wherein the nickase comprises at least one RNA-guided endonuclease having a single active endonuclease domain, thereby forming a nick within each first and each second target DNA sequence; and (c) contacting the double-stranded DNA sample with a strand-displacing polymerase and one or more nucleotides, thereby forming a single-stranded flap on the double-stranded DNA sample beginning at each nick of step (b), wherein each single-stranded flap hybridizes to its corresponding complementary strand of the double stranded DNA sample, thereby generating linked-paired-end DNA fragments.

In some embodiments, the first target DNA sequence and the second target DNA sequence of each sgRNA pair is located adjacent to a protospacer adjacent motif (PAM) sequence.

In some embodiments, the method further comprises inactivating the nickase(s).

In some embodiments, the sgRNA library is computationally designed to target sequences within the double-stranded DNA sample.

In some embodiments, the first target DNA sequence and the second target DNA sequence are separated by about 50 to about 1000 base pairs (bp) of the double-stranded DNA sample.

In some embodiments, each linked-paired-end DNA fragment comprises a linker sequence at each end of the DNA fragment, wherein each linker sequence comprises from about 50 to about 1000 bp of DNA sequence which is at least 90%, at least 95%, at least 98%, at least 99%, or at least 100% identical to a linker sequence of an adjacent DNA fragment.

In some embodiments, the sgRNA library comprises at least 5, at least 10, at least 25, at least 50, at least 100, at least 250, at least 500, at least 600, at least 700, at least 800, at least 900, or at least 1000 distinct sgRNAs.

In some embodiments, obtaining the sgRNA library comprises synthesizing the sgRNA library in a single reaction.

In some embodiments, synthesizing the multiple sgRNAs in a single reaction comprises: (i) obtaining a dsDNA duplex library wherein each dsDNA duplex comprises a T7 promoter sequence operably linked to a sequence encoding an sgRNA, and further wherein the dsDNA duplex library is treated with exonuclease, preferably at about 37° C. for about 1 hour, and purified to remove single-stranded DNA (ssDNA); (ii) contacting the dsDNA duplex library of step (i) with T7 RNA polymerase and NTPs, preferably at about 37° C. for about 2 hours, thereby synthesizing the sgRNA library; (iii) contacting the dsDNA duplex library of step (ii) with DNase I, preferably at about 37° C. for about 15 minutes, thereby degrading the dsDNA duplexes; and (iv) optionally purifying and/or quantifying the sgRNA library.

In some embodiments, the RNA-guided endonuclease is a clustered regularly interspaced short palindromic repeat (CRISPR)-associated endonuclease selected from a Cas9 and a Cas12a (Cpf1).

In some embodiments, the RNA-guided endonuclease is D10A Cas9 or H840A Cas9.

In some embodiments, the strand-displacing polymerase comprises Klenow Fragment or D141A/E143A Thermococcus litoralis (“Vent exo-”) DNA polymerase.

In some embodiments, the linked-paired-end DNA fragments range in size from about 100 bp up to about 1,000,000 bp (1 Mbp) or more.

In some embodiments, the linked-paired-end DNA fragments range in size from about 100 bp up to about 20,000 bp.

In some embodiments, the linked-paired-end DNA fragments are uniformly spaced within the double-stranded DNA sample.

In some embodiments, the double-stranded DNA sample comprises at least one genome selected from a viral genome, a bacterial genome, an archaeal genome, a fungal genome, a plant genome, an animal genome, a mammalian genome, and a human genome.

In some embodiments, the double-stranded DNA sample comprises a mixture of genomes, wherein the mixture of genomes comprises at least two genomes and up to about about 50, about 100, about 500, about 1000, about 2000, or about 3000 or more genomes.

In some embodiments, the method further comprises modifying the generated linked-paired-end DNA fragments with repair enzymes, 3′-deoxyadenosine (dA) tail addition, and/or adapter ligation.

In some embodiments, the generated linked-paired-end DNA fragments are further processed such that each linked-paired-end DNA fragment is 5′-phosphorylated and comprises a 3′-dA tail.

In some embodiments, the method further comprises (a) circularizing the linked-paired-end fragments, (b) fragmenting the circularized fragments, (c) size selecting the fragments of interest from step (b), and ligating adapters to the fragments of interest.

In some embodiments, each of the generated linked-paired-end DNA fragments is ligated to a pair of universal adapters and amplified by long-range PCR.

In some embodiments, the method further comprises sequencing the generated linked-paired-end DNA fragments with a high throughput sequencing platform.

In some embodiments, the high throughput sequencing platform is selected from the group consisting of Illumina sequencing, SOLiD sequencing, 454 pyrosequencing, Ion Torrent semiconductor sequencing, single molecule real-time (SMRT) circular consensus sequencing, and nanopore (MinION) sequencing.

In some embodiments, the high throughput sequencing platform is nanopore (MinION) sequencing.

According to a second aspect of the invention, a method of preparing a DNA sequencing library comprising DNA fragments having linked-paired ends from at least one double-stranded DNA sample having a first and a second DNA strand is provided, the method comprising: (a) obtaining a single guide RNA (sgRNA) library comprising multiple sgRNAs, wherein each sgRNA targets a first target DNA sequence on the first DNA strand; (b) contacting the double-stranded DNA sample with the sgRNA library and at least one first nickase, wherein the first nickase comprises at least one RNA-guided endonuclease having a single active endonuclease domain, thereby forming a nick within each first target DNA sequence; (c) contacting the double-stranded DNA sample with at least one second nickase, wherein the second nickase comprises a nicking restriction endonuclease which targets a second target DNA sequence on the second DNA strand, thereby forming a nick within each second target DNA sequence, wherein step (b) and step (c) may be performed in any order or simultaneously; and (d) contacting the double-stranded DNA sample with a strand-displacing polymerase and one or more nucleotides, thereby forming a single-stranded flap on the double-stranded DNA sample beginning at each nick of steps (b) and (c), wherein each single-stranded flap hybridizes to its corresponding complementary strand of the double stranded DNA sample, thereby generating linked-paired-end DNA fragments.

In some embodiments, the first target DNA sequence of each sgRNA is located adjacent to a protospacer adjacent motif (PAM) sequence.

In some embodiments, the nicking restriction endonuclease comprises one or more endonucleases selected from the group consisting of Nb.BbvCI, Nt.BbvCI, Nt.Bsml, Nt.BsmAI, Nt.BstNBI, Nb.BsrDI, Nb.BstI, Nt.BspQI, Nt.BpulOI and Nt.Bpul0I.

In some embodiments, the method further comprises inactivating the nickase(s).

In some embodiments, the sgRNA library is computationally designed to target sequences within the double-stranded DNA sample.

In some embodiments, the first target DNA sequence and the second target DNA sequence are separated by about 50 to about 1000 base pairs (bp) of the double-stranded DNA sample.

In some embodiments, each linked-paired-end DNA fragment comprises a linker sequence at each end of the DNA fragment, wherein each linker sequence comprises from about 50 to about 1000 bp of DNA sequence which is at least 90%, at least 95%, at least 98%, at least 99%, or at least 100% identical to a linker sequence of an adjacent DNA fragment.

In some embodiments, the sgRNA library comprises at least 5, at least 10, at least 25, at least 50, at least 100, at least 250, at least 500, at least 600, at least 700, at least 800, at least 900, or at least 1000 distinct sgRNAs.

In some embodiments, obtaining the sgRNA library comprises synthesizing the sgRNA library in a single reaction.

In some embodiments, synthesizing the multiple sgRNAs in a single reaction comprises: (i) obtaining a dsDNA duplex library wherein each dsDNA duplex comprises a T7 promoter sequence operably linked to a sequence encoding an sgRNA, and further wherein the dsDNA duplex library is treated with exonuclease, preferably at about 37° C. for about 1 hour, and purified to remove single-stranded DNA (ssDNA); (ii) contacting the dsDNA duplex library of step (i) with T7 RNA polymerase and NTPs, preferably at about 37° C. for about 2 hours, thereby synthesizing the sgRNA library; (iii) contacting the dsDNA duplex library of step (ii) with DNase I, preferably at about 37° C. for about 15 minutes, thereby degrading the dsDNA duplexes; and (iv) optionally purifying and/or quantifying the sgRNA library.

In some embodiments, the sgRNA library is generated on a surface of a substrate using single stranded (ss)oligonucleotides. In some embodiments, the substrate is glass.

In some embodiments, the ss oligonucleotides are synthesized directly on the surface using photolithography.

In some embodiments, about one million sgRNAs can be simultaneously generated on the surface.

In some embodiments, the RNA-guided endonuclease is a clustered regularly interspaced short palindromic repeat (CRISPR)-associated endonuclease selected from a Cas9 and a Cas12a (Cpf1).

In some embodiments, the RNA-guided endonuclease is D10A Cas9 or H840A Cas9.

In some embodiments, the strand-displacing polymerase comprises Klenow Fragment or D141A/E143A Thermococcus litoralis (“Vent exo-”) DNA polymerase.

In some embodiments, the linked-paired-end DNA fragments range in size from about 100 bp up to about 1,000,000 bp (1 Mbp) or more.

In some embodiments, the linked-paired-end DNA fragments range in size from about 100 bp up to about 20,000 bp.

In some embodiments, the linked-paired-end DNA fragments are uniformly spaced within the double-stranded DNA sample.

In some embodiments, the double-stranded DNA sample comprises at least one genome selected from a viral genome, a bacterial genome, an archaeal genome, a fungal genome, a plant genome, an animal genome, a mammalian genome, and a human genome.

In some embodiments, the double-stranded DNA sample comprises a mixture of genomes, wherein the mixture of genomes comprises at least two genomes and up to about about 50, about 100, about 500, about 1000, about 2000, or about 3000 or more genomes.

In some embodiments, the method further comprises modifying the generated linked-paired-end DNA fragments with repair enzymes, 3′-deoxyadenosine (dA) tail addition, and/or adapter ligation.

In some embodiments, the generated linked-paired-end DNA fragments are further processed such that each linked-paired-end DNA fragment is 5′-phosphorylated and comprises a 3′-dA tail.

In some embodiments, the method further comprises (a) circularizing the linked-paired-end fragments, (b) fragmenting the circularized fragments, (c) size selecting the fragments of interest from step (b), and ligating adapters to the fragments of interest.

In some embodiments, each of the generated linked-paired-end DNA fragments is ligated to a pair of universal adapters and amplified by long-range PCR.

In some embodiments, the method further comprises sequencing the generated linked-paired-end DNA fragments with a high throughput sequencing platform.

In some embodiments, the high throughput sequencing platform is selected from the group consisting of Illumina sequencing, SOLiD sequencing, 454 pyrosequencing, Ion Torrent semiconductor sequencing, single molecule real-time (SMRT) circular consensus sequencing, and nanopore (MinION) sequencing.

In some embodiments, the high throughput sequencing platform is nanopore (MinION) sequencing.

According to a third aspect of the invention, a method of generating at least one de novo whole genome map is provided, the method comprising: (a) sequencing the DNA sequencing library prepared by a method disclosed herein with a high throughput sequencing platform, thereby generating sequence reads; and (b) computationally processing the sequence reads to align adjacent linker sequences, thereby ordering the linked-paired-end DNA fragments and generating the at least one de novo whole genome map.

In some embodiments, the sequencing comprises at least 10× sequencing coverage.

In some embodiments, computationally processing the sequence reads further comprises correlating the sequence reads to a sequence assembly, a genetic or cytogenetic map, a structural pattern, a structural variation, a physiological characteristic, a methylation pattern, an epigenomic pattern, a location of a CpG island, a single nucleotide polymorphism (SNP), a copy number variation (CNV), or a combination thereof.

In some embodiments, the processing further comprises assembly of a haplotype sequence.

In some embodiments, the haplotype sequence comprises a major histocompatibility (MHC) region of a mammalian genome, preferably a human genome.

According to a fourth aspect, the invention provides a microdevice for generating both a sgRNA library and a DNA sequencing library, wherein the device comprises a first substrate having a first surface; and a plurality of recessed portions extending from the first surface into the first substrate, wherein each of the plurality of the recessed portions comprises either a microwell or a micro flow channel.

In some embodiments, each of the plurality of microwells is used for generating either the sgRNA library or for generating the DNA sequencing library.

In some embodiments, each of the plurality of microwells used for generating the sgRNA library is in fluidic communication with at least one microwell used for generating the DNA sequencing library.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates the steps of a method for synthesizing sgRNAs according to an embodiment of the invention.

FIG. 2 is a schematic illustrating an embodiment of the invention for creating double-stranded DNA fragments having linker sequences on either end that, when sequenced, facilitate the identification and alignment of adjacent fragments. This method preserves linkage identity, enables haplotyping and facilitates de novo sequences assembly by contig joining. Specifically, H840A Cas9 nickase is used with an sgRNA library targeting DNA target sequence pairs which are in a (+/−) orientation. The DNA target sequences of each pair are adjacent to a PAM, are separated by about 50 to about 1000 bp, and generate linker sequences of the same length as the separation distance (i.e., about 50 to about 1000 bp) upon further processing with a strand-displacing polymerase. Notably, use of D10A Cas9 with an sgRNA library targeting DNA target sequence pairs which are in a (+/−) orientation does not produce any DNA fragments. Further, extension with Taq polymerase results in production of fragments which do not comprise linker sequences.

FIG. 3 is a schematic illustrating an embodiment of the invention for creating double-stranded DNA fragments having linker sequences on either end that, when sequenced, facilitate the identification and alignment of adjacent fragments. This method preserves linkage identity, enables haplotyping and facilitates de novo sequences assembly by contig joining. Specifically, D10A Cas9 nickase is used with an sgRNA library targeting DNA target sequence pairs which are in a (−/+) orientation. The DNA target sequences of each pair are adjacent to a PAM, are separated by about 50 to about 1000 bp, and generate linker sequences of the same length as the separation distance (i.e., about 50 to about 1000 bp) upon further processing with a strand-displacing polymerase. Notably, use of H840A Cas9 with an sgRNA library targeting DNA target sequence pairs which are in a (−/+) orientation does not produce any DNA fragments. Further, extension with Taq polymerase results in production of fragments which do not comprise linker sequences.

FIG. 4A illustrates the fragment sizes and linker sequence sizes for Lambda DNA fragmentation with H840A Cas9 and an sgRNA library targeting DNA target sequence pairs which are in a (+/−) orientation.

FIG. 4B illustrates the fragment sizes and linker sequence sizes for Lambda DNA fragmentation with D10A Cas9 and an sgRNA library targeting DNA target sequence pairs which are in a (−/+) orientation.

FIG. 5 provides a gel showing data related to fragmentation of Lambda genomic DNA.

FIG. 6 provides a gel showing data related to fragmentation of Lambda genomic DNA

FIG. 7 provides nanopore sequencing reads aligned to the Lambda DNA reference.

FIG. 8 provides a magnified view of nanopore sequencing data of two fragmentation sites of Lambda genomic DNA.

FIG. 9 provides a gel showing long-range PCR of Lambda DNA fragments after two-step ligation.

FIG. 10 is a schematic showing steps for selectively preparing sequencing samples containing target structural variants (SVs) to be sequenced, while dephosphorylating and blocking the non-target DNA fragments.

FIG. 11 is a histogram of read length vs base called bases for 100 human genes that were sequenced according to the embodiments presented herein.

FIGS. 12A-12B are tables showing the details regarding the design for guide RNAs for sequencing both long and short human genes and experimental results of sequencing those genes, respectively. The results show that 100 (out of 103) human genes were accurately sequenced using the methods according to the embodiments presented herein.

FIG. 13 provides nanopore sequencing reads for RNF43 gene.

FIG. 14 provides magnified view of the sequencing reads of FIG. 13.

FIG. 15 is a schematic for on-surface sgRNA synthesis using oligos.

FIG. 16 is a representation of a micro-device, which comprises chambers/microwells for both guide RNA synthesis as well as for generating the sequencing library.

### DETAILED DESCRIPTION OF THE INVENTION

The present invention relates to innovative means of DNA mapping and sequencing technology based on massively parallel sequencing with linked-paired-end sequencing libraries. Thus, in various embodiments described herein, the methods of the invention relate to methods of generating paired-end nucleic acid fragment sharing common linker nucleic acid sequences using a nicking endonuclease (nickase) comprising an RNA-guided endonuclease and optionally, a nicking restriction enzyme, methods of analyzing the nucleotides sequences from the linked-paired-end sequenced fragments and methods of de novo whole genome mapping.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

The term, “polynucleotide” includes cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semisynthetic nucleotide bases. Also, included within the scope of the invention are alterations of a wild type or synthetic gene, including but not limited to deletion, insertion, substitution of one or more nucleotides, or fusion to other polynucleotide sequences.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The term “oligonucleotide” or “oligos” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” or “protein” are used interchangeably, and refer to a compound comprised of amino acid residues covalently linked by peptide bonds. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs and fusion proteins, among others. The polypeptides include natural peptides, recombinant peptides, synthetic peptides or a combination thereof. A peptide that is not cyclic will have a N-terminal and a C-terminal. The N-terminal will have an amino group, which may be free (i.e., as a NH2 group) or appropriately protected (for example, with a BOC or a Fmoc group). The C-terminal will have a carboxylic group, which may be free (i.e., as a COOH group) or appropriately protected (for example, as a benzyl or a methyl ester). A cyclic peptide does not have free N- or C-terminal, since they are covalently bonded through an amide bond to form the cyclic structure. Amino acids may be represented by their full names (for example, leucine), 3-letter abbreviations (for example, Leu) and 1-letter abbreviations (for example, L). The structure of amino acids and their abbreviations may be found in the chemical literature, such as in Stryer, “Biochemistry”, 3rd Ed., W. H. Freeman and Co., New York, 1988. tLeu represents tert-leucine. neo-Trp represents 2-amino-3-(1H-indol-4-y)-propanoic acid. DAB is 2,4-diaminobutyric acid. Orn is ornithine. N-Me-Arg or N-methyl-Arg is 5-guanidino-2-(methylamino) pentanoic acid.

“Sample” or “biological sample” as used herein means a biological material from a subject, including but is not limited to organ, tissue, cell, exosome, blood, plasma, saliva, urine and other body fluid, A sample can be any source of material obtained from a subject.

The terms “subject”, “patient”, “individual”, and the like are used interchangeably herein, and refer to any animal, or cells thereof whether in vitro or in situ, amenable to the methods described herein. In certain non-limiting embodiments, the patient, subject or individual is a human. Non-human mammals include, for example, livestock and pets, such as ovine, bovine, porcine, canine, feline and murine mammals. Preferably, the subject is human. The term “subject” does not denote a particular age or sex.

The term “measuring” according to the present invention relates to determining the amount or concentration, preferably semi-quantitatively or quantitatively. Measuring can be done directly.

As used herein the term “amount” refers to the abundance or quantity of a constituent in a mixture.

The term “concentration” refers to the abundance of a constituent divided by the total volume of a mixture. The term concentration can be applied to any kind of chemical mixture, but most frequently it refers to solutes and solvents in solutions.

As used herein, the terms “reference”, or “threshold” are used interchangeably, and refer to a value that is used as a constant and unchanging standard of comparison.

As used herein, “paired-end sequencing” is a sequencing method that is based on high throughput sequencing in which both ends of a DNA fragment are sequenced. Any high throughput DNA sequencing platform may be used, such as those based on the platforms currently sold by Illumina, Oxford Nanopore, Pacific Biosciences, and Roche. Oxford Nanopore's MinION sequencer can generate short to ultra-long (>2 Mb) reads. Illumina has released a hardware module (the PE Module) which can be installed in an existing sequencer as an upgrade, which allows sequencing of both ends of the template, thereby generating paired end reads. Paired end sequencing may also be conducted using Solexa, Oxford Nanopore, or PacBio single-molecule real-time (SMRT) circular consensus sequencing (CCS) technology in the methods according to the current invention. Examples of paired end sequencing are described for instance in US20060292611 and in publications from Roche (454 sequencing).

As used herein the term “sequencing” refers to determining the order of nucleotides (base sequences) in a nucleic acid sample, e.g. DNA or RNA. Many techniques are available such as Sanger sequencing and high-throughput sequencing technologies (also known as next-generation sequencing technologies) such as pyrosequencing based on the “sequencing by synthesis” principle, in which the sequencing is performed by detecting the nucleotide incorporated by a DNA polymerase. Pyrosequencing generally relies on light detection based on a chain reaction when pyrophosphate is released.

A “restriction endonuclease” or “restriction enzyme” refers to an enzyme that recognizes a specific nucleotide sequence (target site) in a double-stranded DNA molecule, and will cleave both strands of the DNA molecule at or near every target site, leaving a blunt or a staggered end.

A “Type-IIs” restriction endonuclease refers to an endonuclease that has a recognition sequence that is distant from the restriction site. In other words, Type IIs restriction endonucleases cleave outside of the recognition sequence to one side. Examples thereof are NmeAlll (GCCGAG(21/19)) and FokI, AlwI, Mme I. Also included in this definition are Type IIs enzymes that cut outside the recognition sequence at both sides.

A “Type IIb” restriction endonuclease cleaves DNA at both sides of the recognition sequence.

“Restriction fragments” or “DNA fragments” refer to DNA molecules produced by digestion of DNA with a restriction endonuclease are referred to as restriction fragments. Any given genome (or nucleic acid, regardless of its origin) can be digested by a particular restriction endonuclease into a discrete set of restriction fragments. The DNA fragments that result from restriction endonuclease cleavage can be further used in a variety of techniques and can, for instance, be detected by gel electrophoresis or sequencing. Restriction fragments can be blunt ended or have an overhang. The overhang can be removed using a technique described as polishing. The term ‘internal sequence’ of a restriction fragment is typically used to indicate that the origin of the part of the restriction fragment resides in the sample genome, i.e. does not form part of an adapter. The internal sequence is directly derived from the sample genome, its sequence is hence part of the sequence of the genome under investigation.

As used herein, “Ligation” refers to the enzymatic reaction catalyzed by a ligase enzyme in which two double-stranded DNA molecules are covalently joined together. In general, both DNA strands are covalently joined together, but it is also possible to prevent the ligation of one of the two strands through chemical or enzymatic modification of one of the ends of the strands. In that case, the covalent joining will occur in only one of the two DNA strands.

“Adapters” or “adaptors” are short double-stranded DNA molecules with a limited number of base pairs, e.g. about 10 to about 30 base pairs in length, which are designed such that they can be ligated to the ends of DNA fragments, such as the linked-paired-end DNA fragments generated by the methods described herein. Adapters are generally composed of two synthetic oligonucleotides that have nucleotide sequences which are partially complementary to each other. When mixing the two synthetic oligonucleotides in solution under appropriate conditions, they will anneal to each other forming a double-stranded structure. After annealing, one end of the adapter molecule is designed such that it is compatible with the end of a DNA fragment and can be ligated thereto; the other end of the adapter can be designed so that it cannot be ligated, but this need not be the case (double ligated adapters). Adapters can contain other functional features such as identifiers, recognition sequences for restriction enzymes, primer binding sections etc. When containing other functional features the length of the adapters may increase, but by combining functional features this may be controlled.

“Adapter-ligated DNA fragments” refer to DNA fragments that have been capped by adapters on one or both ends.

As used herein, “barcode” or “tag” refer to a short sequence that can be added or inserted to an adapter or a primer or included in its sequence or otherwise used as label to provide a unique barcode (aka barcode or index). Such a sequence barcode (tag) can be a unique base sequence of varying but defined length, typically from 4-16 bp used for identifying a specific nucleic acid sample. For instance 4 bp tags allow 4 4=256 different tags. Using such an barcode, the origin of a PCR sample can be determined upon further processing or fragments can be related to a clone. Also clones in a pool can be distinguished from one another using these sequence based barcodes. Thus, barcodes can be sample specific, pool specific, clone specific, amplicon specific etc. In the case of combining processed products originating from different nucleic acid samples, the different nucleic acid samples are generally identified using different barcodes. Barcodes preferably differ from each other by at least two base pairs and preferably do not contain two identical consecutive bases to prevent misreads. The barcode function can sometimes be combined with other functionalities such as adapters or primers and can be located at any convenient position. A barcode is often used as a fingerprint for labeling a DNA fragment and/or a library and for constructing a multiplex library. The library includes, but not limited to, genomic DNA library, cDNA library and ChIP library. Libraries, of which each is separately labeled with a distinct barcode, may be pooled together to form a multiplex barcoded library for performing sequencing simultaneously, in which each barcode is sequenced together with its flanking tags located in the same construct and thereby serves as a fingerprint for the DNA fragment and/or library labeled by it. A “barcode” is positioned in between two restriction enzyme (RE) recognition sequences. A barcode may be virtual, in which case the two RE recognition sites themselves become a barcode. Preferably, a barcode is made with a specific nucleotide sequence having 0 (i.e., a virtual sequence), 1, 2, 3, 4, 5, 6, or more base pairs in length. The length of a barcode may be increased along with the maximum sequencing length of a sequencer.

As used herein, “primers” refer to DNA strands which can prime the synthesis of DNA. DNA polymerase cannot synthesize DNA de novo without primers: it can only extend an existing DNA strand in a reaction in which the complementary strand is used as a template to direct the order of nucleotides to be assembled. The synthetic oligonucleotide molecules which are used in a polymerase chain reaction (PCR) as primers are referred to as “primers”.

As used herein, the term “DNA amplification” will be typically used to denote the in vitro synthesis of double-stranded DNA molecules using PCR. It is noted that other amplification methods exist and they may be used in the present invention without departing from the gist.

As used herein, “aligning” means the comparison of two or more nucleotide sequences based on the presence of short or long stretches of identical or similar nucleotides. Several methods for alignment of nucleotide sequences are known in the art, as will be further explained below.

“Alignment” refers to the positioning of multiple sequences in a tabular presentation to maximize the possibility for obtaining regions of sequence identity across the various sequences in the alignment, e.g. by introducing gaps. Several methods for alignment of nucleotide sequences are known in the art, as will be further explained below.

The term “contig” is used in connection with DNA sequence analysis, and refers to assembled contiguous stretches of DNA derived from two or more DNA fragments having contiguous nucleotide sequences. Thus, a contig is a set of overlapping DNA fragments that provides a partial contiguous sequence of a genome. A “scaffold” is defined as a series of contigs that are in the correct order, but are not connected in one continuous sequence, i.e. contain gaps. Contig maps also represent the structure of contiguous regions of a genome by specifying overlap relationships among a set of clones. For example, the term “contigs” encompasses a series of cloning vectors which are ordered in such a way as to have each sequence overlap that of its neighbors. The linked clones can then be grouped into contigs, either manually or, preferably, using appropriate computer programs such as FPC, PHRAP, CAP3 etc.

“Fragmentation” refers to a technique used to fragment DNA into smaller fragments. Fragmentation can be enzymatic, chemical or physical. Random fragmentation is a technique that provides fragments with a length that is independent of their sequence. Typically, shearing or nebulisation are techniques that provide random fragments of DNA. Typically, the intensity or time of the random fragmentation is determinative for the average length of the fragments. Following fragmentation, a size selection can be performed to select the desired size range of the fragments

“Physical mapping” describes techniques using molecular biology techniques such as hybridization analysis, PCR and sequencing to examine DNA molecules directly in order to construct maps showing the positions of sequence features.

“Genetic mapping” is based on the use of genetic techniques such as pedigree analysis to construct maps showing the positions of sequence features on a genome

The term “genome”, as used herein, relates to a material or mixture of materials, containing genetic material from an organism. The term “genomic DNA” as used herein refers to deoxyribonucleic acids that are obtained from an organism or which are derived from an RNA genome such as a viral genome. The terms “genome” and “genomic DNA” encompass genetic material that may have undergone amplification, purification, or fragmentation.

The term “reference genome”, as used herein, refers to a sample comprising genomic DNA to which a test sample may be compared. In certain cases, reference genome contains regions of known sequence information.

The term “double-stranded” as used herein refers to nucleic acids formed by hybridization of two single strands of nucleic acids containing complementary sequences. In most cases, genomic DNA are double-stranded.

As used herein, the term “single nucleotide polymorphism”, or “SNP” for short, refers to single nucleotide position in a genomic sequence for which two or more alternative alleles are present at appreciable frequency (e.g., at least 1%) in a population.

The term “chromosomal region” or “chromosomal segment”, as used herein, denotes a contiguous length of nucleotides in a genome of an organism. A chromosomal region may be in the range of 1000 nucleotides in length to an entire chromosome, e.g., 100 kb to 10 MB for example.

The terms “sequence alteration” or “sequence variation”, as used herein, refer to a difference in nucleic acid sequence between a test sample and a reference sample that may vary over a range of 1 to 10 bases, 10 to 100 bases, 100 to 100 kb, or 100 kb to 10 MB. Sequence alteration may include single nucleotide polymorphism and genetic mutations relative to wild-type. In certain embodiments, sequence alteration results from one or more parts of a chromosome being rearranged within a single chromosome or between chromosomes relative to a reference. In certain cases, a sequence alteration may reflect a difference, e.g. abnormality, in chromosome structure, such as an inversion, a deletion, an insertion or a translocation relative to a reference chromosome, for example.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2,7, 3, 4, 5, 5.3, and 6. This applies regardless of the breadth of the range.

As used herein, the term “endonuclease” refers to enzymes which cleave a phosphodiester bond within a polynucleotide chain (for example, enzymes which have an activity described as EC 3.1.21, EC 3.1.22, or EC 3.1.25, according to the IUBMB enzyme nomenclature).

“Site-specific endonucleases”, also known as “restriction endonucleases” or “restriction enzymes” recognize specific nucleotide sequences in double-stranded DNA. Generally, endonucleases cleave both DNA strands of a DNA duplex. Some sequence-specific endonucleases can be engineered and/or modified to comprise only a single active endonuclease domain which cleaves only one of the strands in a DNA duplex and are thus referred to herein as “nicking endonucleases” or “nicking restriction endonucleases”. Nicking endonuclease catalyzes the hydrolysis of a phosphodiester bond, resulting in either a 5′ or 3′ phosphomonoester. Examples of nicking restriction endonucleases, such as those available from New England Biolabs, include Nb.BbvCI, Nt.BbvCI, Nt.Bsml, Nt.BsmAI, Nt.BstNBI, Nb.BsrDI, Nb.BstI, Nt.BspQI, Nt.BpulOI and Nt.Bpul0I. The cleavage site or “nick site” of the phosphodiester backbone may fall within or outside of the recognition sequence, such as immediately adjacent the recognition sequence, of the site-specific nicking endonuclease.

An “RNA-guided endonuclease” includes those of the CRISPR-Cas (clustered regularly interspaced short palindromic repeats-(CRISPR) associated) adaptive immune systems found in roughly 50% of bacteria and 90% of archaea, as described, e.g., in Jiang and Doudna, Curr Opin Struct Biol. (2015) February; 30:100-111 and Wright et al., Cell (2016) 164(1-2):29-44. RNA-guided endonucleases, such as Cas9, comprise two endonuclease domains. The HNH domain cleaves the target DNA strand whereas the RuvC domain cleaves the non-target DNA strand as defined by a so called “crRNA” strand bound by the endonuclease. According to certain aspects of the invention, the crRNA strand is generally comprised within a single-guide RNA (sgRNA).

As used herein, “nickase” refers to an enzyme which comprises a single active endonuclease domain which cleaves a single strand of DNA within a DNA duplex. In some embodiments, the nickase may be a mutant or variant form of a restriction endonuclease or of an RNA-guided endonuclease. For example, the nickase generally comprises an inactive endonuclease domain which does not cleave DNA, such as D10A Cas9 nickase, H840A Cas9 nickase, and the nicking restriction endonucleases such as Nb.BbvCI, Nt.BbvCI, Nt.Bsml, Nt.BsmAI, Nt.BstNBI, Nb.BsrDI, Nb.BstI, Nt.BspQI, Nt.BpulOI and Nt.Bpul0I.

As used herein, “single guide RNA” or “sgRNA” refers to a single chimeric RNA which comprises the functions of a CRISPR RNA (crRNA) and a trans-acting crRNA known as tracrRNA (trRNA). The DNA cleavage site(s) of an RNA-guided endonuclease are within targeted DNA sequences defined by a 20 nt sequence within the sgRNA and adjacent to a PAM sequence within the DNA, as described in Jinek et al., Science (2012) 337:816-821.

### Description

The present invention relates to innovative methods of DNA mapping based on massively parallel sequencing of linked-paired-end DNA sequencing libraries. In various embodiments, these methods comprise fragmenting a double-stranded DNA sample, such as a DNA sample comprising one or more whole genomes, so that the ends of the adjacent DNA fragments share the same sequences (referred to herein as linker sequences). These linked DNA fragments are then sequenced, and the sequence reads can then be computationally aligned and assembled to generate one or more de novo genome maps and/or mapped back to one or more reference genome maps and assembled. In some embodiments, the double-stranded DNA sample comprises at least one genome selected from a viral genome, a bacterial genome, an archaeal genome, a fungal genome, a plant genome, an animal genome, a mammalian genome, and a human genome. In some embodiments, the double-stranded DNA sample comprises a mixture of genomes, wherein the mixture of genomes comprises at least two genomes and up to about 10, about 50, about 100, about 500, about 1000, about 2000, or about 3000 or more genomes. In some embodiments, the double-stranded DNA sample comprises a major histocompatibility (MHC) region of a mammalian genome, preferably a human genome.

In one aspect, the methods of the invention comprise generating linked-paired-end DNA fragments for sequencing at a specific sequence motif where the ends of adjacent DNA fragments share the same sequences (overlapping sequences referred to herein as “linker sequences” or “linking sequences”). These linker sequences can be about 50 to about 1000 bases long. In some embodiments, the method can be used to generate de novo genome maps. In certain aspects, genetic variations found within the overlapping sequences can be used to separate haplotype-resolved reads and generate scaffolds anchored at specific sequence motifs for subsequent de novo based sequence assembly. As such, in various embodiments, the methods of this invention preserve linkage identity, enable haplotype information and facilitate the de novo sequence assembly with short-read shotgun-type sequencing. The present invention enables achieving high-quality, low-cost de novo assembly of complex genomes and capturing various scales of sequence contiguity information.

DNA Sequencing Library Preparation

Methods of preparing a DNA sequencing library are provided wherein the DNA sequencing library comprises DNA fragments having linked-paired ends from at least one double-stranded DNA sample, such as genomic DNA. Each of the methods employs a nicking RNA-guided endonuclease (“nickase”) to generate nicks in the double-stranded DNA at target sequences which are defined by an sgRNA library. In a first aspect, one or more nicking RNA-guided endonucleases such as, for example, D10A Cas9 and/or H840A Cas9 are used. In a second aspect, one or more nicking RNA-guided endonucleases is used in combination with one or more nicking restriction endonucleases. Each of these embodiments is described in more detail infra.

In a first aspect, a method of preparing a DNA sequencing library is provided, wherein the DNA sequencing library comprises DNA fragments having linked-paired ends from at least one double-stranded DNA sample having a first and a second DNA strand. In various embodiments, the method comprises: (a) obtaining a single guide RNA (sgRNA) library comprising multiple sgRNA pairs, wherein: (i) each sgRNA pair comprises a first sgRNA and a second sgRNA, and (ii) the first sgRNA of each sgRNA pair targets a first target DNA sequence on the first DNA strand and the second sgRNA of each sgRNA pair targets a second target DNA sequence on the second DNA strand; (b) contacting the double-stranded DNA sample with the sgRNA library and at least one nickase, wherein the nickase comprises at least one RNA-guided endonuclease having a single active endonuclease domain, thereby forming a nick within each first and each second target DNA sequence; and (c) contacting the double-stranded DNA sample with a strand-displacing polymerase and one or more nucleotides, thereby forming a single-stranded flap on the double-stranded DNA sample beginning at each nick of step (b), wherein each single-stranded flap hybridizes to its corresponding complementary strand of the double stranded DNA sample, thereby generating linked-paired-end DNA fragments. In some embodiments, the first target DNA sequence and the second target DNA sequence of each sgRNA pair are located adjacent to a protospacer adjacent motif (PAM) sequence.

In a second aspect, a method of preparing a DNA sequencing library is provided wherein the DNA sequencing library comprises DNA fragments having linked-paired ends from at least one double-stranded DNA sample having a first and a second DNA strand. In various embodiments, the method comprises: (a) obtaining a single guide RNA (sgRNA) library comprising multiple sgRNAs, wherein each sgRNA targets a first target DNA sequence on the first DNA strand; (b) contacting the double-stranded DNA sample with the sgRNA library and at least one first nickase, wherein the first nickase comprises at least one RNA-guided endonuclease having a single active endonuclease domain, thereby forming a nick within each first target DNA sequence; (c) contacting the double-stranded DNA sample with at least one second nickase, wherein the second nickase comprises a nicking restriction endonuclease which targets a second target DNA sequence on the second DNA strand, thereby forming a nick within each second target DNA sequence, wherein step (b) and step (c) may be performed in any order or simultaneously; and (d) the double-stranded DNA sample with a strand-displacing polymerase and one or more nucleotides, thereby forming a single-stranded flap on the double-stranded DNA sample beginning at each nick of steps (b) and (c), wherein each single-stranded flap hybridizes to its corresponding complementary strand of the double stranded DNA sample, thereby generating linked-paired-end DNA fragments. In some embodiments, the first target DNA sequence of each sgRNA is located adjacent to a protospacer adjacent motif (PAM) sequence

In some embodiments, the methods further comprise inactivating the nickase(s). Inactivation may comprise heating the reaction, for example at about 72° C. or more, for about an hour.

In some aspects of this invention, the linked-paired-end DNA fragments are further processed prior to high-throughput sequencing. For example, in some embodiments, the method further comprises modifying the generated linked-paired-end DNA fragments with repair enzymes, 3′-deoxyadenosine (dA) tail addition, and/or adapter ligation. In some embodiments, the generated linked-paired-end DNA fragments are further processed such that each linked-paired-end DNA fragment is 5′-phosphorylated and comprises a 3′-dA-tail. In some embodiments, the method further comprises circularizing the generated linked-paired-end DNA fragments, fragmenting the circularized fragments, selecting fragments of interest, and ligating adapters to the fragments of interest. In some embodiments, each of the generated linked-paired-end DNA fragments is ligated to a pair of universal adapters and amplified, such as by long-range PCR, and purified by methods known in the art.

RNA-Guided Endonucleases and Nickases

RNA-guided endonucleases include those of the CRISPR-Cas adaptive immune systems found in roughly 50% of bacteria and 90% of archaea, as described, e.g., in Jiang and Doudna, Curr Opin Struct Biol. (2015) February; 30:100-111 and Wright et al., Cell (2016) 164(1-2):29-44. RNA-guided endonucleases, such as S. pyogenes (sp) Cas9, comprise two endonuclease domains. The HNH domain cleaves the target DNA strand whereas the RuvC domain cleaves the non-target DNA strand, as defined by a so called “crRNA” strand bound by the endonuclease. The crRNA strand is comprised within a single-guide RNA (sgRNA), as described in Jinek et al., Science (2012) 337:816-821. In some embodiments, each sgRNA comprises a 20 nt target sequence located 5′ and adjacent to a NGG PAM sequence followed by a Cas9 recognition sequence.

In some embodiments, suitable nickases are derived from RNA-guided endonucleases comprising a single active endonuclease domain which cleaves a single strand of DNA within a DNA duplex, such as a mutant or variant form an RNA-guided endonuclease. For example, in some embodiments, the nickase comprises an inactive endonuclease domain which does not cleave DNA, such as D10A Cas9 nickase, which has an inactivated RuvC domain and cleaves only the target DNA strand, or H840A Cas9 nickase, which has an inactivated HNH domain and cleaves only the non-target DNA strand. Such nickases bind RNA, such as sgRNA, which defines the targeted sequence within the DNA.

Table 1 provides additional examples of suitable RNA-guided endonucleases and their (PAM) sequences from which suitable nickases may be derived using well-known methods, such as site-directed mutagenesis, to inactivate a single endonuclease domain.

TABLE 1
RNA-guided endonucleases and their associated PAM sequences

| Species/Variant of Cas9 | PAM Sequence* |
|---|---|
| Streptococcus pyogenes (SP); SpCas9 | 3′ NGG |
| SpCas9 D1135E variant | 3′ NGG (reduced NAG binding) |
| SpCas9 VRER variant | 3′ NGCG (SEQ ID NO: 174) |
| SpCas9 EQR variant | 3′ NGAG (SEQ ID NO: 175) |
| SpCas9 VQR variant | 3′ NGAN (SEQ ID NO: 176) or |
| | NGNG (SEQ ID NO: 177) |
| xCas9 | 3′ NG, GAA, or GAT |
| SpCas9-NG | 3′ NG |
| Staphylococcus aureus (SA); SaCas9 | 3′ NNGRRT (SEQ ID NO: 164) or |
| | NNGRR(N) SEQ ID NO: 178 |
| Acidaminococcus sp. (AsCpf1) and | 5′ TTTV (SEQ ID NO: 165) |
| Lachnospiraceae bacterium (LbCpf1) | |
| AsCpf1 RR variant | 5′ TYCV (SEQ ID NO: 166) |
| LbCpf1 RR variant | 5′ TYCV (SEQ ID NO: 167) |
| AsCpf1 RVR variant | 5′ TATV(SEQ ID NO: 168) |
| Campylobacter jejuni (CJ) | 3′ NNNNRYAC(SEQ ID NO: 169) |
| Neisseria meningitidis (NM) | 3′ NNNNGATT(SEQ ID NO: 170) |
| Streptococcus thermophilus (ST) | 3′ NNAGAAW(SEQ ID NO: 171) |
| Treponema denticola (TD) | 3′ NAAAAC(SEQ ID NO: 172) |
| Additional Cas9s from various | PAM sequence may not be |
| species | characterized |

*In the table above, 3′ and 5′ indicate on which end of targeted sequence the PAM is located.

Nicking Restriction Endonucleases

In some embodiments, the restriction endonuclease nickases include, but are not limited to, Nb.BbvCI, Nb.BsmI, NbBsrDI, Nb.BtsI, Nt.AlwI, Nt.BbvCI, Nt.BsmAI, Nt.BspQI, Nt.BstNBI, and Nt.CviPII, used either alone or in various combinations. These and other suitable nicking restriction endonucleases are available from commercial sources, including New England Biolabs and Fermentas. The recognition sequences vary from one to the other and are well known in the art. Some site-specific nicking endonucleases along with their features are summarized herein.

The nickase Nb.BbvCI is derived from an E. coli strain expressing an altered form of the BbvCI restriction genes [Ra+:Rb(E177G)] from Bacillus brevis.

The nickase Nb.BsmI is derived from an E. coli strain that carries the cloned BsmI gene from Bacillus stearothermophilus NUB 36.

The nickase Nb.BsrDI is derived from an E. coli strain expressing only the large subunit of the BsrDI restriction gene from Bacillus stearothermophilus D70.

The nickase Nb.BtsI is derived from an E. coli strain expressing only the large subunit of the BtsI restriction gene from Bacillus thermoglucosidasius.

The nickase Nt.AlwI is an engineered derivative of AlwI which catalyzes a single-strand break four bases beyond the 3′ end of the recognition sequence on the top strand. It is derived from an E. coli strain containing a chimeric gene encoding the DNA recognition domain of AlwI and the cleavage/dimerization domain of Nt.BstNBI.

The nickase Nt.BbvCI is derived from an E. coli strain expressing an altered form of the BbvCI restriction genes [Ra(K169E):Rb+] from Bacillus brevis.

The nickase Nt.BsmAI is derived from an E. coli strain expressing an altered form of the BsmAI restriction genes from Bacillus stearothermophilus A664.

The nickase Nt.BspQI is derived from an E. coli strain expressing an engineered BspQI variant from BspQI restriction enzyme.

The nickase Nt.BstNBI catalyzes a single strand break four bases beyond the 3′ side of the recognition sequence. It is derived from an E. coli strain that carries the cloned Nt.BstNBI gene from Bacillus stearothermophilus 33M.

The nickase Nt.CviPII cleaves one strand of a double-stranded DNA substrate. The final product on pUC19 (a plasmid cloning vector) is an array of bands from 25 to 200 base pairs. CCT is cut less efficiently than CCG and CCA, and some of the CCT sites remain uncleaved. It is derived from an E. coli strain that expresses a fusion of Mxe GyrA intein, chitin-binding domain and a truncated form of the Nt.CviPII nicking endonuclease gene from Chlorella virus NYs-1.

In some embodiments, more than one site-specific nicking endonuclease, e.g. two, three, or more different types of site-specific nicking endonucleases are used. In some specific embodiments, a site-specific nicking endonuclease that does not have any variable nucleotide adjacent to its nick site such as Nt.BbvCI or Nb. BbvCI is used.

In certain embodiments, the nicking is suitably effected at one or more sequence-specific locations, although the nicking can be effected at one or more non-specific locations, including random or non-specific locations.

Strand Extension

After forming nicks in the double-stranded DNA sample according to the methods described herein, strand extension is performed by a strand-displacing polymerase. Not wishing to be bound by theory, it is postulated that the strand-displacing polymerase synthesizes a new strand beginning at each nick in the 5′ to 3′ direction and displaces the original strand, wherein the original strand forms a flap. The DNA fragments are then broken off between the opposite strand across from the flap junction to generate two DNA fragments. Each fragment contains “sticky ends” or “overhangs”, which are then filled by the polymerase by incorporation of replacement nucleotides such that the final fragments are blunt-ended and the ends of the two adjacent fragments share the same sequence, referred to herein as a linker sequence. The incorporation of these replacement nucleotides can be conceptualized as filling-in the gap left behind by the formation and “peeling-up” of the flap. By filling in the gap, the position formerly occupied by the flap is occupied by a sequence of bases that suitably has the same sequence as the bases located in the flap. The filling prevents re-hybridization of the flap to the second stand of DNA to which the flap was formerly bound.

In some embodiments, the generated flap is about 1 to about 1000 bases in length. Typically, a flap is from about 50 to about 1000 bases, or from about 20 to about 500 bases in length, or even in the range of from about 30 to about 50 bases.

In further embodiments, the strand extension involves one or more strand-displacing polymerases, such as Klenow fragment (which lacks 5′ to 3′ exonuclease activity) or D141A/E143A Thermococcus litoralis (Vent® (exo-) polymerase (which lacks 3′ to 5′ exonuclease activity) and a nucleotide composition to accommodate the various needs. In certain cases, the nucleotide composition facilitates multi-color labeling, in which there may be at least two, three, or four distinguishably labeled nucleotides. In further cases, the detectable label of a nucleotide comprises a tag that emits a color or a non-fluorescent tag that is further processed for visualization. In yet further embodiments, the nucleotide mixture comprises phosphorothioated nucleotides, e.g., nucleoside alpha-thiotriphosphates (also known as alpha-thionucleoside triphosphates).

Single-Guide RNA (sgRNA) Libraries

According to various aspects of the invention, single-guide RNA (sgRNA) libraries are computationally designed to target specific sequences within the double-stranded DNA sample using methods which are well-known in the art. Examples of suitable algorithms and tools for designing sgRNAs are described in Cui et al., Interdisciplinary Sciences: Computational Life Sciences (2018) 10:455-465. In some embodiments, the target sequences are generally designed to be uniformly spaced within a genome or double-stranded DNA sample and/or the sgRNAs are generally designed to minimize off-target nicking. Suitable target sequences are generally 20 nt long and appropriately adjacent to a PAM sequence, for example, 5′ to a NGG PAM sequence. In some embodiments, sgRNA pairs are designed wherein a first sgRNA targets a first target sequence on the first DNA strand and a second sgRNA targets a second target sequence on the second DNA strand, and further wherein the first target sequence and the second target sequences are spaced about 50 to about 1000 bp apart. The first and second target sequences are selected based on the locations of PAM sequences in the double-stranded DNA sample, such as a genome. As such, the sgRNA pairs are designed such that they target sequences which are in either a (+/−) or a (−/+) orientation. The (+/−) orientation indicates that the first PAM site and first target sequence on the first DNA strand is located upstream of the second PAM site and second target sequence on the second DNA strand. The (−/+) orientation analogously indicates that the first PAM site and first target sequence on the first DNA strand is located downstream of the second PAM site and second target sequence on the second DNA strand. In some embodiments, H840A Cas9 is used in combination with a (+/−) sgRNA library. In some embodiments, D10A Cas9 is used in combination with a (−/+) sgRNA library. In some embodiments, sgRNAs are designed to target a PAM-adjacent sequence which is about 50 to about 1000 bp away from and either upstream or downstream from a nicking restriction endonuclease recognition sequence on the opposite DNA strand. In such embodiments, an RNA-guided nickase is used in combination with a nicking restriction endonuclease.

Synthesis of sgRNA libraries may be performed by any method known in the art. For example, the method described by Gagon et al. (vol 9, e98186, 2014) Plos One, 9 may be used. In some embodiments, the sgRNA library is synthesized in a single reaction, that is, in a single reaction tube, although a single vessel, well, and/or droplet may alternatively be used, such that all sgRNAs within the library are synthesized simultaneously without the need for a separate reaction for each sgRNA. In some embodiments, the sgRNA library comprises up to several hundred sgRNAs. In some embodiments, the sgRNA library comprises at least 5, at least 10, at least 25, at least 50, at least 100, at least 250, at least 500, at least 600, at least 700, at least 800, at least 900, or at least 1000 distinct sgRNAs.

In some embodiments, the sgRNA library is synthesized in a single reaction by a method comprising (i) obtaining a dsDNA duplex library wherein each dsDNA duplex comprises a T7 promoter sequence operably linked to a sequence encoding an sgRNA, and further wherein the dsDNA duplex library is treated with exonuclease, preferably at about 37° C. for about 1 hour, and purified to remove single-stranded DNA (ssDNA); (ii) contacting the dsDNA duplex library of step (i) with T7 RNA polymerase and NTPs, preferably at about 37° C. for about 2 hours, thereby synthesizing the sgRNA library; (iii) contacting the dsDNA duplex library of step (ii) with DNase I, preferably at about 37° C. for about 15 minutes, thereby degrading the dsDNA duplexes; and (iv) optionally purifying and/or quantifying the sgRNA library.

In some embodiments, each dsDNA duplex comprising a T7 promoter sequence operably linked to a sequence encoding an sgRNA is generated from (i) a first ssDNA oligo comprising, from 5′ to 3′, a T7 promoter sequence, a 20 nt target sequence, and an “overlap” sequence of about 10 nt to about 20 nt and (ii) a second ssDNA oligo comprising from 3′ to a 10 to 20 nt sequence complementary to the “overlap” sequence and a longer sequence of about 65 nt which will be a template strand for the sgRNA synthesis. The two ssDNA oligos are hybridized and extended by DNA polymerase to form the dsDNA duplex which is transcribed by RNA polymerase to generate the sgRNA. Each sgRNA comprises a guide RNA (target) sequence followed by a Cas9 binding sequence.

In some embodiments, the sgRNA library is synthesized on a surface of a single substrate using single-stranded oligonucleotides. In some embodiments, the substrate is a glass substrate. In some embodiments, the single-stranded oligonucleotides up to 100 nucleotides and one million such oligonucleotides can be synthesized directly on the modified glass surface, in situ, using photolithography. Each synthesized oligonucleotide is similar to the oligonucleotides described elsewhere herein and comprises a promotor sequence, 20 bases of guide (gRNA) target sequence, and an overlapping sequence, which can be hybridized with another universal oligonucleotide. The process of on-surface sgRNA generation is same as that of in-tube sgRNA synthesis as described elsewhere herein. However, about a million sgRNAs can be generated with a single on-surface reaction.

DNA Mapping

The invention includes methods relating to DNA mapping, including methods for making linked-paired-end sequenced genomic DNA fragments, methods of analyzing the nucleotides sequences of the linked fragments and identifying multiple sequence motifs or polymorphic sites, and methods of establishing sequence contiguity across the whole genome. These methods generate continuous base by base sequencing information, within the context of the DNA map allowing de novo whole genome mapping. Compared with prior art methods, the present methods of DNA mapping provide improved sequence contiguity across the whole genome, and achieve high-quality, fast, and low-cost de novo assembly of complex genomes.

In one embodiment, the generated linked-paired-end fragment are directly shotgun sequenced. This sequencing procedure involves diluting the linked-paired-end fragments, amplifying them by PCR and sequencing them.

In another embodiment, the generated linked-paired-end fragment are processed further in a library for sequencing. Various sequencing platforms are known in the art. The choice of a platform may be based on the user's and experiment's requirements. In some embodiments, the sequencing method is a high throughput next-generation method. Non limiting example of massively parallel signature sequencing platforms are MinION sequencing (Oxford Nanopore, UK), Illumina sequencing by synthesis (Illumina, san Diego CA), 454 pyrosequencing (Roche Diagnostics, Indianapolis IN), SOLiD sequencing (Life Technologies, Carlsbad, CA), Ion Torrent semiconductor sequencing (Life Technologies, Carlsbad, CA), Heliscope single molecule sequencing (Helicos Biosciences, Cambridge, MA), and Single molecule real time (SMRT) circular consensus sequencing (Pacific Biosciences, Menlo Park, CA). In some embodiments, due to the length of the linker sequences, only about 10× sequencing coverage is sufficient.

In certain aspects of the invention, the library preparation for sequencing comprises the following main steps: (a) circularizing the paired-end linked fragments, (b) fragmenting, (c) size selecting the fragments of interest, and (d) ligating adapters at one or both end(s) of the fragments for single or paired-end sequencing. In further aspects, known barcoded nucleotide adapters are incorporated to the adapters ligation step (d). In other aspects, the sequencing library construction and adapters/barcodes addition increases both sides of the linked-paired-end fragments by 50, 100, 150, 200 or more bases.

In another embodiment, the sequenced linked-paired-end fragments of the invention are useful for whole genome mapping. In certain embodiment, the method allows efficient (about 20 times) enrichment of the target genes from a genome. In certain embodiment, the method comprises sequencing the entire gene including exons and introns. In certain aspects, the linked-paired end fragments are computationally aligned based on the overlapping linker sequences and appropriately arranged to generate de novo whole genome maps. In other aspects, by determining the positions of the sequenced linkers/adapters within each fragment with respect to a reference known genomic DNA backbone, the distribution of the linked-paired-end fragments can be mapped accurately base by base and assembled. This method is illustrated elsewhere herein in the identification of lambda phage DNA molecules. In yet another embodiment, the sequenced linked-paired-end fragments of the invention are useful for haplotype-scaffold-sequencing (HSS) wherein the sequence contiguity across the whole genome is established allowing de novo haplotype sequence assembly of haploid human genomes. In a further embodiment, the haplotype sequence assembly comprises the human major histocompatibility (MHC) region.

In another embodiment, the sequencing information from the linked-paired-end fragments allow a broad range of computational analysis of the sequence reads. The wide variety of analysis can be appreciated and performed by those skilled in the art. Non-limiting examples where the sequenced linked-paired-end fragments are used include capturing various scales of sequence and structural variation, haplotypes, methylation pattern, epigenomic pattern, location of CpG islands, single nucleotide polymorphisms (SNPs), copy number variations (CNVs), introns retentions and other nucleotides configurations for coding and non-coding elements.

Devices

In one aspect, the invention provides a microdevice such that both sgRNA library and a DNA sequencing library are generated within the micro device, wherein the device comprises a first substrate having a first surface and a plurality of recessed portions extending from the first surface into the first substrate.

In some embodiments, the recessed portion is either a microwell or a micro flow channel. In some embodiments, each of plurality of microwells is used for generating either the sgRNA library or for generating the DNA sequencing library.

In some embodiments, each of the plurality of microwells used for generating the sgRNA library is in fluidic communication with at least one microwell used for generating the DNA sequencing library such that sgRNAs from the microwell can be transported to the well wherein DNA sequencing library is being generated.

In another aspect, the invention provides a device having a surface for preparing sgRNA library. In some embodiments, the sgRNA library is synthesized on the surface using single-stranded oligonucleotides. In some embodiments, the single-stranded oligonucleotides up to 100 nucleotides and one million such oligonucleotides can be synthesized directly on the surface, in situ, using photolithography techniques. Each synthesized oligonucleotide is similar to oligonucleotides described elsewhere herein and comprises a promotor sequence, 20 bases of guide (gRNA) target sequence, and an overlapping sequence, which can be hybridized with another universal oligonucleotide. The process of on-surface sgRNA generation is same as that of in-tube sgRNA synthesis as described elsewhere herein. However, a million sgRNAs can be generated with a single on-surface reaction. As an example, approximately 40,000 sgRNAs for sequencing the whole exome can be generated at once on the surface. Likewise, approximately 150,000 sgRNAs for sequencing the whole human genome can be synthesized at once on the surface.

The methods and devices presented herein can be used for various applications such as, for example, target-sequencing including gene panels, whole exome sequencing, whole genome sequencing, and microbe sequencing

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in the experiments disclosed herein are now described.

Materials and Methods

Lambda DNA is from New England BioLabs (NEB). D10A Cas9, nicking restriction enzymes, Klenow polymerase, Taq Polymerase, T7 Endonuclease, Taq ligase and other enzymes are from NEB. H840A Cas9 and DNA oligos are from Integrated DNA technology (IDT). Single-stranded flap sequences are introduced by incubating nicked DNA with certain polymerases which lack 5′-3′ or 3′-5′ exonuclease activity such as Klenow (Exo-) polymerase or Vent® (exo-) polymerase. In cases where the DNA was fragmented with a Cas9 nickase in combination with a restriction nickase, BSPQI nickase was employed to nick the opposite strand.

DNA samples were assessed by running electrophoresis using a 1% agarose gel slab in 1×TAE buffer at 110 V for 75 minutes. DNA was stained with 1×SYBRSafe stain (Thermoscientific).

### Example 1: sgRNA Library Synthesis

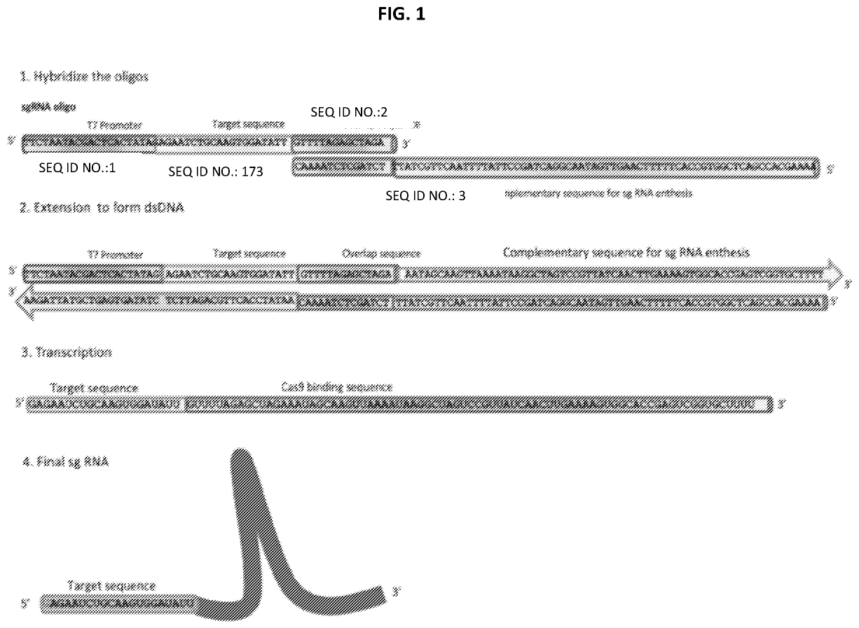

A library of ssDNA oligomers each having a T7 promoter sequence (5′-TTCTAATACGACTCACTATAG-3′) (SEQ ID NO: 1), a 20-mer guide RNA sequence (target sequence), and an “overlap” sequence (5′-GTTTTAGAGCTAGA-3′) (SEQ ID NO: 2), were designed and ordered from IDT. These oligos were hybridized with a second ssDNA oligo comprising a segment for Cas9 binding and a segment which is complementary to the overlap sequence which facilitates hybridization (5′-AAAAGCACCGACTCGGTGCCACTTTTTAAGTTGATAACGGACTAGCCTTATTTTA ACTTGCTATTTCTAGCTCTAAAAC-3′) (SEQ ID NO: 3). The hybridized oligos were then extended to form dsDNA which were then purified and used as templates for a subsequent transcription reaction in which the sgRNA were generated as shown in FIG. 1. Notably, the extension/hybridization and transcription reactions of the library can each be carried out in a single reaction, such as a single reaction tube, vessel, well or droplet. These sgRNA were used in Cas9-mediated modification reactions.

Briefly, a hybridization reaction was carried out in 1× Buffer 2(NEB). 10 uM of designed oligomers and 10 uM of a common complementary overlap sequence containing oligomer were first denatured at 95° C. for 15 s and allowed to hybridize at 43° C. for 5 min. The hybridized oligos were then extended with 5 U of Klenow exo- at 37° C. for 1 h in the presence of 2 mM dNTPs.

Next, an exonuclease treatment was carried out at 37° C. for 1 h with 10 U of exonuclease I (NEB) in 1× exonuclease buffer (NEB). The dsDNA was then purified using Qiagen Nucleotide removal kit and assessed later using Synergy H1 plate reader (Biotek).

A transcription reaction was then carried out on the purified and quantified dsDNA using the T7 HiScribe transcription kit (NEB). The T7 RNA Polymerase recognizes the T7 promoter region that seeds transcription of the adjacent 20-mer target sequence thus generating the sgRNA for targets in the Cas9-mediated nicking.

The synthesized sgRNA were purified using Monarch RNA purification kit (NEB) and assessed using Synergy H1 plate reader (Biotek). Purified dsDNA and sgRNA were stored at −20° C. and found to be viable for at least 3 weeks in absence of any contamination.

Guide RNA (target) sequences, as well as the ssDNA oligos used for generating sgRNAs comprising the target sequences, are shown in Tables 2-4.

TABLE 2
Guide RNA and ssDNA Oligos for Lambda DNA with H840A Cas9

| | | Linker | |
|---|---|---|---|
| Strand | Location | Length | 20-mer gRNA (target) sequence for H840A Cas9 |
| + | 6,525 | | GCAGTTTCTGCCGTGCTTAA (SEQ ID NO: 4) |
| - | 6,738 | 213 | CGGAACAGCGCCCAGCCTTT (SEQ ID NO: 5) |
| + | 13,144 | | TTCGGTCCCTTCTGTAAGAA (SEQ ID NO: 6) |
| - | 13,263 | 119 | CAGAAACGACTCCAGTACCG (SEQ ID NO: 7) |
| + | 24,629 | | CTGTAGCTGCTGAAACGTTG (SEQ ID NO: 8) |
| - | 24,719 | 90 | ACAGGTATCGTTTGGAGGCA (SEQ ID NO: 9) |
| + | 26,560 | | AGTTACCCCTCTAAGTAATG (SEQ ID NO: 10) |
| - | 27,154 | 594 | CCATGCAACATGAATAACAG (SEQ ID NO: 11) |
| + | 33,817 | | TTTCCTCTGTCATTACGTCA (SEQ ID NO: 12) |
| - | 34,261 | 444 | CGACTATTGATAAAAATCAA (SEQ ID NO: 13) |
| + | 47,566 | | ATGTTTTCACTTAATAGTAT (SEQ ID NO: 14) |
| - | 47,703 | 137 | TGCGCTTGCTCTTCATCTAG (SEQ ID NO: 15) |
| | | Linker | |
| Strand | Location | Length | ssDNA Oligo |
| + | 6,525 | | TTCTAATACGACTCACTATAGGCAGTTTCTGCCGTGCTTAAGTT |
| | | | TTAGAGCTAGA (SEQ ID NO: 16) |
| - | 6,738 | 213 | TTCTAATACGACTCACTATAGCGGAACAGCGCCCAGCCTTTGT |
| | | | TTTAGAGCTAGA (SEQ ID NO: 17) |
| + | 13,144 | | TTCTAATACGACTCACTATAGTTCGGTCCCTTCTGTAAGAAGTT |
| | | | TTAGAGCTAGA (SEQ ID NO: 18) |
| - | 13,263 | 119 | TTCTAATACGACTCACTATAGCAGAAACGACTCCAGTACCGGT |
| | | | TTTAGAGCTAGA (SEQ ID NO: 19) |
| + | 24,629 | | TTCTAATACGACTCACTATAGCTGTAGCTGCTGAAACGTTGGTT |
| | | | TTAGAGCTAGA (SEQ ID NO: 20) |
| - | 24,719 | 90 | TTCTAATACGACTCACTATAGACAGGTATCGTTTGGAGGCAGT |
| | | | TTTAGAGCTAGA (SEQ ID NO: 21) |
| + | 26,560 | | TTCTAATACGACTCACTATAGAGTTACCCCTCTAAGTAATGGTT |
| | | | TTAGAGCTAGA (SEQ ID NO: 22) |
| − | 27,154 | 594 | TTCTAATACGACTCACTATAGCCATGCAACATGAATAACAGGT |
| | | | TTTAGAGCTAGA (SEQ ID NO: 23) |
| + | 33,817 | | TTCTAATACGACTCACTATAGTTTCCTCTGTCATTACGTCAGTT |
| | | | TTAGAGCTAGA (SEQ ID NO: 24) |
| - | 34,261 | 444 | TTCTAATACGACTCACTATAGCGACTATTGATAAAAATCAAGT |
| | | | TTTAGAGCTAGA (SEQ ID NO: 25) |
| + | 47,566 | | TTCTAATACGACTCACTATAGATGTTTTCACTTAATAGTATGTT |
| | | | TTAGAGCTAGA (SEQ ID NO: 26) |
| - | 47,703 | 137 | TTCTAATACGACTCACTATAGTGCGCTTGCTCTTCATCTAGGTT |
| | | | TTAGAGCTAGA (SEQ ID NO: 27) |

TABLE 3
Guide RNA and ssDNA Oligos for Lambda DNA with D10A Cas9

| | Loca- | Linker | |
|---|---|---|---|
| Strand | tion | Length | 20-mer gRNA (target) sequence for D10A Cas9 |
| - | 4,062 | | CCAGCCAGCACAGAAACATC (SEQ ID NO: 28) |
| + | 5,057 | 995 | AGCGGCAGCCATAAGGTGGA (SEQ ID NO: 29) |
| - | 13,087 | | AGGTCTTCATCGTCCACCTC (SEQ ID NO: 30) |
| + | 13,144 | 57 | TTCGGTCCCTTCTGTAAGAA (SEQ ID NO: 31) |
| - | 24,566 | | TGAATGACTTCCCCAATTAT (SEQ ID NO: 32) |
| + | 24,629 | 63 | CTGTAGCTGCTGAAACGTTG (SEQ ID NO: 33) |
| - | 26,436 | | TGATTTAACTATACCTTTTG (SEQ ID NO: 34) |
| + | 27,221 | 785 | CGCCGAACGATTAGCTCTTC (SEQ ID NO: 35) |
| - | 34,261 | | CGACTATTGATAAAAATCAA (SEQ ID NO: 36) |
| + | 34,478 | 217 | CAGTTTGATGAGTATAGAAA (SEQ ID NO: 37) |
| - | 47,443 | | GAAGGTTTTACCAATGGCTC (SEQ ID NO: 38) |
| + | 47,566 | 123 | ATGTTTTCACTTAATAGTAT (SEQ ID NO: 39) |
| | Loca- | Linker | |
| Strand | tion | Length | ssDNA Oligo |
| - | 4,062 | | TTCTAATACGACTCACTATAGCCAGCCAGCACAGAAACATCGT |
| | | | TTTAGAGCTAGA (SEQ ID NO: 40) |
| + | 5,057 | 995 | TTCTAATACGACTCACTATAGAGCGGCAGCCATAAGGTGGAGT |
| | | | TTTAGAGCTAGA (SEQ ID NO: 41) |
| - | 13,087 | | TTCTAATACGACTCACTATAGAGGTCTTCATCGTCCACCTCGTT |
| | | | TTAGAGCTAGA (SEQ ID NO: 42) |
| + | 13,144 | 57 | TTCTAATACGACTCACTATAGTTCGGTCCCTTCTGTAAGAAGTT |
| | | | TTAGAGCTAGA (SEQ ID NO: 43) |
| - | 24,566 | | TTCTAATACGACTCACTATAGTGAATGACTTCCCCAATTATGTT |
| | | | TTAGAGCTAGA (SEQ ID NO: 44) |
| + | 24,629 | 63 | TTCTAATACGACTCACTATAGCTGTAGCTGCTGAAACGTTGGTT |
| | | | TTAGAGCTAGA (SEQ ID NO: 45) |
| - | 26,436 | | TTCTAATACGACTCACTATAGTGATTTAACTATACCTTTTGGTT |
| | | | TTAGAGCTAGA (SEQ ID NO: 46) |
| + | 27,221 | 785 | TTCTAATACGACTCACTATAGCGCCGAACGATTAGCTCTTCGTT |
| | | | TTAGAGCTAGA (SEQ ID NO: 47) |
| - | 34,261 | | TTCTAATACGACTCACTATAGCGACTATTGATAAAAATCAAGT |
| | | | TTTAGAGCTAGA (SEQ ID NO: 48) |
| + | 34,478 | 217 | TTCTAATACGACTCACTATAGCAGTTTGATGAGTATAGAAAGT |
| | | | TTTAGAGCTAGA (SEQ ID NO: 49) |
| - | 47,443 | | TTCTAATACGACTCACTATAGGAAGGTTTTACCAATGGCTCGTT |
| | | | TTAGAGCTAGA (SEQ ID NO: 50) |
| + | 47,566 | 123 | TTCTAATACGACTCACTATAGATGTTTTCACTTAATAGTATGTT |
| | | | TTAGAGCTAGA (SEQ ID NO: 51) |

TABLE 4
Guide RNA and ssDNA Oligos for H. influenzae NP3311 DNA with D10A Cas9

| | | Linker | |
|---|---|---|---|
| Strand | Location | Length | 20-mer gRNA (target) sequence for D10A Cas9 |
| - | 1,122,507 | | TATGCACCGCCAGTATAAGT (SEQ ID NO: 52) |
| + | 1,122,699 | 192 | AAAAATAATGTTGCATCAAT (SEQ ID NO: 53) |
| - | 1,140,119 | | GTCCTTCTCGTTAAAAAATC (SEQ ID NO: 54) |
| + | 1,140,351 | 232 | TGCTATCAATGATTCCCGCT (SEQ ID NO: 55) |
| - | 1,164,071 | | GAAAAACCTGATGTTTACAT (SEQ ID NO: 56) |
| + | 1,164,488 | 417 | TCCGCAATTTGCTCAATTTC (SEQ ID NO: 57) |
| - | 1,171,068 | | TCGTCATGCTCAATGGCGTT (SEQ ID NO: 58) |
| + | 1,171,398 | 330 | AAGACCAAATTTCAAAGTCA (SEQ ID NO: 59) |
| - | 1,178,293 | | GACTGGGGATTATTCGCAGG (SEQ ID NO: 60) |
| + | 1,178,487 | 194 | AACTTGGTTACCATCCCAAT (SEQ ID NO: 61) |
| - | 1,200,881 | | AATGATGTTGAATTCCAAGT (SEQ ID NO: 62) |
| + | 1,201,270 | 389 | TGCATTGCGAGGATTAGCAA (SEQ ID NO: 63) |
| - | 1,228,941 | | AAGAATAAAAGTGGCCAAAT (SEQ ID NO: 64) |
| + | 1,229,352 | 411 | GCTGTGCCGTTGTTTGTATT (SEQ ID NO: 65) |
| - | 1,260,860 | | CAATTTTTAGATCGCTTACG (SEQ ID NO: 66) |
| + | 1,261,206 | 346 | TGCGTAATAATTGTCCGCTT (SEQ ID NO: 67) |
| - | 1,290,900 | | GGCATTCAAGATATTATCAC (SEQ ID NO: 68) |
| + | 1,291,304 | 404 | TAGGAGGTTTGCGAACTACG (SEQ ID NO: 69) |
| - | 1,321,761 | | CCCGTATCCTTTGGTGCGGT (SEQ ID NO: 70) |
| + | 1,322,145 | 384 | CAAGGTAAGGCAACATAAGA (SEQ ID NO: 71) |
| - | 1,338,898 | | CCAAACGTAACTTGCTTAAT (SEQ ID NO: 72) |
| + | 1,339,104 | 206 | CATAATTTCCGCCTTTTATT (SEQ ID NO: 73) |
| - | 1,358,417 | | GATGATATGATTGATACTGG (SEQ ID NO: 74) |
| + | 1,358,810 | 393 | TGGCGAGCATAGCCGAAATA (SEQ ID NO: 75) |
| - | 1,364,031 | | TATAAAATTATTGAATGGGT (SEQ ID NO: 76) |
| + | 1,364,409 | 378 | ATAGGTAAGAATAAACCACG (SEQ ID NO: 77) |
| - | 1,378,763 | | CATGATGAACCGTGAGAGAG (SEQ ID NO: 78) |
| + | 1,379,074 | 311 | TCAAACAGTTAATTTGAGTA (SEQ ID NO: 79) |
| - | 1,393,657 | | GCGATAATTAAAACTAAAAT (SEQ ID NO: 80) |
| + | 1,393,879 | 222 | GTGGGAATTAAATCAATGTC (SEQ ID NO: 81) |
| - | 1,407,866 | | CTTGAAAAAATTATCGCAGC (SEQ ID NO: 82) |
| + | 1,408,210 | 344 | GAGCACCACCTTGACATGGT (SEQ ID NO: 83) |
| - | 1,421,673 | | GAGAATTAATACGATAGCCT (SEQ ID NO: 84) |
| + | 1,422,070 | 397 | GGTCGCCGTCAAATCGATTT (SEQ ID NO: 85) |
| - | 1,435,001 | | ACTCTCATTAGAGACGTTTT (SEQ ID NO: 86) |
| + | 1,435,345 | 344 | CCTGCCGGTCGCAAGATTGT (SEQ ID NO: 87) |
| - | 1,448,525 | | TTTTGTGCCTGCGTATTTGT (SEQ ID NO: 88) |
| + | 1,448,847 | 322 | TGATTTTATCAATGGCAAGG (SEQ ID NO: 89) |
| - | 1,461,970 | | TTCCGGCGTATCCGCCCAAG (SEQ ID NO: 90) |
| + | 1,462,406 | 436 | TGGAGGTGCTCAAGTTATGT (SEQ ID NO: 91) |
| - | 1,475,429 | | ATAAACACTTCCCCACTACT (SEQ ID NO: 92) |
| + | 1,475,689 | 260 | TGGTGGGGAACGTCAGCGTG (SEQ ID NO: 93) |
| - | 1,491,310 | | ATTGATGAAAAACCAATTGG (SEQ ID NO: 94) |
| + | 1,491,588 | 278 | GTTTTTATTCGTGTAATATA (SEQ ID NO: 95) |
| - | 1,504,867 | | GAGGTTTAATATGTCTAAAG (SEQ ID NO: 96) |
| + | 1,505,340 | 473 | TTAGGTACAGTTATCCGTGG (SEQ ID NO: 97) |
| - | 1,524,636 | | TTTTTTCTTTTGTTCTTTAG (SEQ ID NO: 98) |
| + | 1,525,112 | 476 | GTTGTTTTAAACGAAAAATG (SEQ ID NO: 99) |
| - | 1,546,785 | | AATTTAGTGCCTGCATTTAA (SEQ ID NO: 100) |
| + | 1,547,000 | 215 | TTGATAAGAATCGCCAATAT (SEQ ID NO: 101) |
| - | 1,563,404 | | CATATTTCTGTAAAATATTG (SEQ ID NO: 102) |
| + | 1,563,684 | 280 | GCAGAACGTTATATCGGCGG (SEQ ID NO: 103) |
| - | 1,575,680 | | GGGCGCAAAATTCAATCAGG (SEQ ID NO: 104) |
| + | 1,576,074 | 394 | GTCGGTTCGAGTCCGACCCT (SEQ ID NO: 105) |
| - | 1,601,517 | | AATTGGCCGCACTCACTTAA (SEQ ID NO: 106) |
| + | 1,601,956 | 439 | AATTTCATGTGGCATTGATG (SEQ ID NO: 107) |
| | | Linker | |
| Strand | Location | Length | ssDNA Oligo |
| - | 1,122,507 | | TTCTAATACGACTCACTATAGTATGCACCGCCAGTATAAGTG |
| | | | TTTTAGAGCTAGA (SEQ ID NO: 108) |
| + | 1,122,699 | 192 | TTCTAATACGACTCACTATAGAAAAATAATGTTGCATCAATG |
| | | | TTTTAGAGCTAGA (SEQ ID NO: 109) |
| - | 1,140,119 | | TTCTAATACGACTCACTATAGGTCCTTCTCGTTAAAAAATCGT |
| | | | TTTAGAGCTAGA (SEQ ID NO: 110) |
| + | 1,140,351 | 232 | TTCTAATACGACTCACTATAGTGCTATCAATGATTCCCGCTGT |
| | | | TTTAGAGCTAGA (SEQ ID NO: 111) |
| - | 1,164,071 | | TTCTAATACGACTCACTATAGGAAAAACCTGATGTTTACATG |
| | | | TTTTAGAGCTAGA (SEQ ID NO: 112) |
| + | 1,164,488 | 417 | TTCTAATACGACTCACTATAGTCCGCAATTTGCTCAATTTCGT |
| | | | TTTAGAGCTAGA (SEQ ID NO: 113) |
| - | 1,171,068 | | TTCTAATACGACTCACTATAGTCGTCATGCTCAATGGCGTTGT |
| | | | TTTAGAGCTAGA (SEQ ID NO: 114) |
| + | 1,171,398 | 330 | TTCTAATACGACTCACTATAGAAGACCAAATTTCAAAGTCAG |
| | | | TTTTAGAGCTAGA (SEQ ID NO: 115) |
| - | 1,178,293 | | TTCTAATACGACTCACTATAGGACTGGGGATTATTCGCAGGG |
| | | | TTTTAGAGCTAGA (SEQ ID NO: 116) |
| + | 1,178,487 | 194 | TTCTAATACGACTCACTATAGAACTTGGTTACCATCCCAATGT |
| | | | TTTAGAGCTAGA (SEQ ID NO: 117) |
| - | 1,200,881 | | TTCTAATACGACTCACTATAGAATGATGTTGAATTCCAAGTG |
| | | | TTTTAGAGCTAGA (SEQ ID NO: 118) |
| + | 1,201,270 | 389 | TTCTAATACGACTCACTATAGTGCATTGCGAGGATTAGCAAG |
| | | | TTTTAGAGCTAGA (SEQ ID NO: 119) |
| - | 1,228,941 | | TTCTAATACGACTCACTATAGAAGAATAAAAGTGGCCAAATG |
| | | | TTTTAGAGCTAGA (SEQ ID NO: 120) |
| + | 1,229,352 | 411 | TTCTAATACGACTCACTATAGGCTGTGCCGTTGTTTGTATTGT |
| | | | TTTAGAGCTAGA (SEQ ID NO: 121) |
| - | 1,260,860 | | TTCTAATACGACTCACTATAGCAATTTTTAGATCGCTTACGGT |
| | | | TTTAGAGCTAGA (SEQ ID NO: 122) |
| + | 1,261,206 | 346 | TTCTAATACGACTCACTATAGTGCGTAATAATTGTCCGCTTGT |
| | | | TTTAGAGCTAGA (SEQ ID NO: 123) |
| - | 1,290,900 | | TTCTAATACGACTCACTATAGGGCATTCAAGATATTATCACG |
| | | | TTTTAGAGCTAGA (SEQ ID NO: 124) |
| + | 1,291,304 | 404 | TTCTAATACGACTCACTATAGTAGGAGGTTTGCGAACTACGG |
| | | | TTTTAGAGCTAGA (SEQ ID NO: 125) |
| - | 1,321,761 | | TTCTAATACGACTCACTATAGCCCGTATCCTTTGGTGCGGTGT |
| | | | TTTAGAGCTAGA (SEQ ID NO: 126) |
| + | 1,322,145 | 384 | TTCTAATACGACTCACTATAGCAAGGTAAGGCAACATAAGAG |
| | | | TTTTAGAGCTAGA (SEQ ID NO: 127) |
| - | 1,338,898 | | TTCTAATACGACTCACTATAGCCAAACGTAACTTGCTTAATG |
| | | | TTTTAGAGCTAGA (SEQ ID NO: 128) |
| + | 1,339,104 | 206 | TTCTAATACGACTCACTATAGCATAATTTCCGCCTTTTATTGT |
| | | | TTTAGAGCTAGA (SEQ ID NO: 129) |
| - | 1,358,417 | | TTCTAATACGACTCACTATAGGATGATATGATTGATACTGGG |
| | | | TTTTAGAGCTAGA (SEQ ID NO: 130) |
| + | 1,358,810 | 393 | TTCTAATACGACTCACTATAGTGGCGAGCATAGCCGAAATAG |
| | | | TTTTAGAGCTAGA (SEQ ID NO: 131) |
| - | 1,364,031 | | TTCTAATACGACTCACTATAGTATAAAATTATTGAATGGGTG |
| | | | TTTTAGAGCTAGA (SEQ ID NO: 132) |
| + | 1,364,409 | 378 | TTCTAATACGACTCACTATAGATAGGTAAGAATAAACCACGG |
| | | | TTTTAGAGCTAGA (SEQ ID NO: 133) |
| - | 1,378,763 | | TTCTAATACGACTCACTATAGCATGATGAACCGTGAGAGAGG |
| | | | TTTTAGAGCTAGA (SEQ ID NO: 134) |
| + | 1,379,074 | 311 | TTCTAATACGACTCACTATAGTCAAACAGTTAATTTGAGTAG |
| | | | TTTTAGAGCTAGA (SEQ ID NO: 135) |
| - | 1,393,657 | | TTCTAATACGACTCACTATAGGCGATAATTAAAACTAAAATG |
| | | | TTTTAGAGCTAGA (SEQ ID NO: 136) |
| + | 1,393,879 | 222 | TTCTAATACGACTCACTATAGGTGGGAATTAAATCAATGTCG |
| | | | TTTTAGAGCTAGA (SEQ ID NO: 137) |
| - | 1,407,866 | | TTCTAATACGACTCACTATAGCTTGAAAAAATTATCGCAGCG |
| | | | TTTTAGAGCTAGA (SEQ ID NO: 138) |
| + | 1,408,210 | 344 | TTCTAATACGACTCACTATAGGAGCACCACCTTGACATGGTG |
| | | | TTTTAGAGCTAGA (SEQ ID NO: 139) |
| - | 1,421,673 | | TTCTAATACGACTCACTATAGGAGAATTAATACGATAGCCTG |
| | | | TTTTAGAGCTAGA (SEQ ID NO: 140) |
| + | 1,422,070 | 397 | TTCTAATACGACTCACTATAGGGTCGCCGTCAAATCGATTTG |
| | | | TTTTAGAGCTAGA (SEQ ID NO: 141) |
| - | 1,435,001 | | TTCTAATACGACTCACTATAGACTCTCATTAGAGACGTTTTGT |
| | | | TTTAGAGCTAGA (SEQ ID NO: 142) |
| + | 1,435,345 | 344 | TTCTAATACGACTCACTATAGCCTGCCGGTCGCAAGATTGTG |
| | | | TTTTAGAGCTAGA (SEQ ID NO: 143) |
| - | 1,448,525 | | TTCTAATACGACTCACTATAGTTTTGTGCCTGCGTATTTGTGT |
| | | | TTTAGAGCTAGA (SEQ ID NO: 144) |
| + | 1,448,847 | 322 | TTCTAATACGACTCACTATAGTGATTTTATCAATGGCAAGGG |
| | | | TTTTAGAGCTAGA (SEQ ID NO: 145) |
| - | 1,461,970 | | TTCTAATACGACTCACTATAGTTCCGGCGTATCCGCCCAAGG |
| | | | TTTTAGAGCTAGA (SEQ ID NO: 146) |
| + | 1,462,406 | 436 | TTCTAATACGACTCACTATAGTGGAGGTGCTCAAGTTATGTG |
| | | | TTTTAGAGCTAGA (SEQ ID NO: 147) |
| - | 1,475,429 | | TTCTAATACGACTCACTATAGATAAACACTTCCCCACTACTGT |
| | | | TTTAGAGCTAGA (SEQ ID NO: 148) |
| + | 1,475,689 | 260 | TTCTAATACGACTCACTATAGTGGTGGGGAACGTCAGCGTGG |
| | | | TTTTAGAGCTAGA (SEQ ID NO: 149) |
| - | 1,491,310 | | TTCTAATACGACTCACTATAGATTGATGAAAAACCAATTGGG |
| | | | TTTTAGAGCTAGA (SEQ ID NO: 150) |
| + | 1,491,588 | 278 | TTCTAATACGACTCACTATAGGTTTTTATTCGTGTAATATAGT |
| | | | TTTAGAGCTAGA (SEQ ID NO: 151) |
| - | 1,504,867 | | TTCTAATACGACTCACTATAGGAGGTTTAATATGTCTAAAGG |
| | | | TTTTAGAGCTAGA (SEQ ID NO: 152) |
| + | 1,505,340 | 473 | TTCTAATACGACTCACTATAGTTAGGTACAGTTATCCGTGGG |
| | | | TTTTAGAGCTAGA (SEQ ID NO: 153) |
| - | 1,524,636 | | TTCTAATACGACTCACTATAGTTTTTTCTTTTGTTCTTTAGGTT |
| | | | TTAGAGCTAGA (SEQ ID NO: 154) |
| + | 1,525,112 | 476 | TTCTAATACGACTCACTATAGGTTGTTTTAAACGAAAAATGG |
| | | | TTTTAGAGCTAGA (SEQ ID NO: 155) |
| - | 1,546,785 | | TTCTAATACGACTCACTATAGAATTTAGTGCCTGCATTTAAGT |
| | | | TTTAGAGCTAGA (SEQ ID NO: 156) |
| + | 1,547,000 | 215 | TTCTAATACGACTCACTATAGTTGATAAGAATCGCCAATATG |
| | | | TTTTAGAGCTAGA (SEQ ID NO: 157) |
| - | 1,563,404 | | TTCTAATACGACTCACTATAGCATATTTCTGTAAAATATTGGT |
| | | | TTTAGAGCTAGA (SEQ ID NO: 158) |
| + | 1,563,684 | 280 | TTCTAATACGACTCACTATAGGCAGAACGTTATATCGGCGGG |
| | | | TTTTAGAGCTAGA (SEQ ID NO: 159) |
| - | 1,575,680 | | TTCTAATACGACTCACTATAGGGGCGCAAAATTCAATCAGGG |
| | | | TTTTAGAGCTAGA (SEQ ID NO: 160) |
| + | 1,576,074 | 394 | TTCTAATACGACTCACTATAGGTCGGTTCGAGTCCGACCCTG |
| | | | TTTTAGAGCTAGA (SEQ ID NO: 161) |
| - | 1,601,517 | | TTCTAATACGACTCACTATAGAATTGGCCGCACTCACTTAAG |
| | | | TTTTAGAGCTAGA (SEQ ID NO: 162) |
| + | 1,601,956 | 439 | TTCTAATACGACTCACTATAGAATTTCATGTGGCATTGATGGT |
| | | | TTTAGAGCTAGA (SEQ ID NO: 163) |

The sgRNA library can also be generated on a single surface of a substrate such as, for example, a glass substrate. Single-stranded oligonucleotides up to 100 nucleotides and about one million such oligonucleotides can be synthesized directly on a modified glass surface using photolithography techniques developed in oligo-microarray technology (Fodor, S. P. et al. (1991) Light-directed, spatially addressable parallel chemical synthesis. 251, 767-773). Each synthesized oligonucleotide is similar to oligonucleotides described elsewhere herein and comprises a promotor sequence, 20 bases of guide (gRNA) target sequence, and an overlapping sequence, which can be hybridized with another universal oligonucleotide. The process of on-surface sgRNA generation is the same as that of in-tube sgRNA synthesis described elsewhere herein. However, a million sgRNAs can be generated with a single on-surface reaction.

### Example 2: Linked DNA Fragmentation of Bacteriophage Lambda Genomic DNA

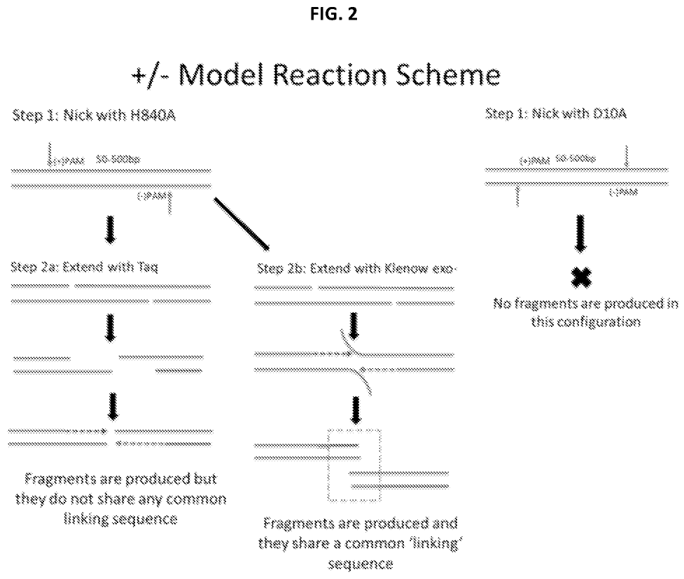

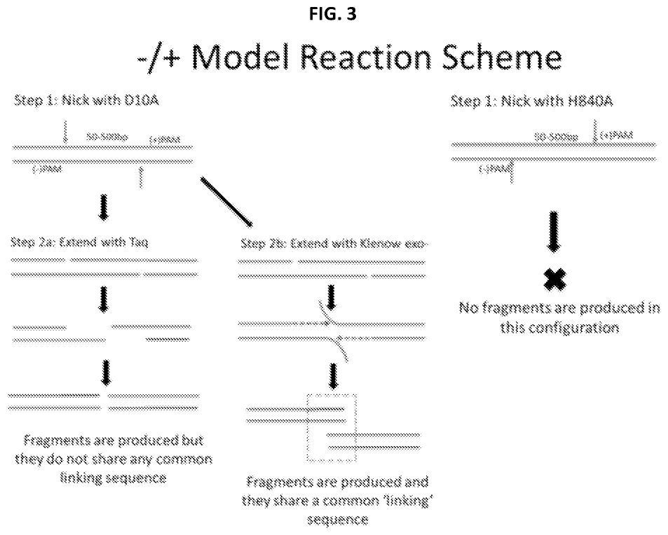

To demonstrate a proof of concept for the linker sequencing library generation, Lambda DNA was used as a template and sgRNA pairs were generated in two configurations based on the first PAM site location (FIG. 2 and FIG. 3). The (+/−) configuration is where PAM site occurs first on positive strand followed by a PAM sequence on negative strand (FIG. 2). The separation between each of the sgRNA forming the pair is 50-1000 bp. Similarly, (−/+) configuration is where PAM first occurs on negative strand followed by PAM on positive strand (FIG. 3).

The (+/−) configuration reactions were performed with Cas9 H840A (IDT) (FIG. 2) and the (−/+) configuration reactions were performed with Cas9 D10A (NEB) (FIG. 3). First, 100 ng of Cas9 Nickase was pre-incubated in 1×NEBuffer 3 (NEB) with 2.5 uM sgRNA at 37° C. for 15 min to incorporate the sgRNAs into the nickase. Then, the DNA (300 ng) was added to the Cas9-sgRNA Complex mix and a nicking reaction was carried out at 37° C. for 2 h. The nickase was then inactivated by raising the temperature to 72° C. for 60 min. The nicked DNA was then extended with 5 U of DNA Klenow (exo-) Polymerase (NEB), 100 nM dNTPs, and 1×NEBuffer 3.1 (NEB) at 37° C. for 60 min.

Reaction schemes for both configurations with two types of mutant Cas9 Nickase enzymes i.e., H840A and D10A are as shown in FIG. 2 and FIG. 3, respectively. Briefly, (+/−) configuration with H840A and (−/+) configuration with D10A successfully produce fragments but when used in any other combination are not successful in fragmentation. Further, extension with Taq polymerase enzyme fragments the DNA without any shared sequences. Extension using a strand displacing enzyme like Klenow exo- or Vent exo- results in DNA fragments with a shared, common sequence at the fragment ends (linker sequences).

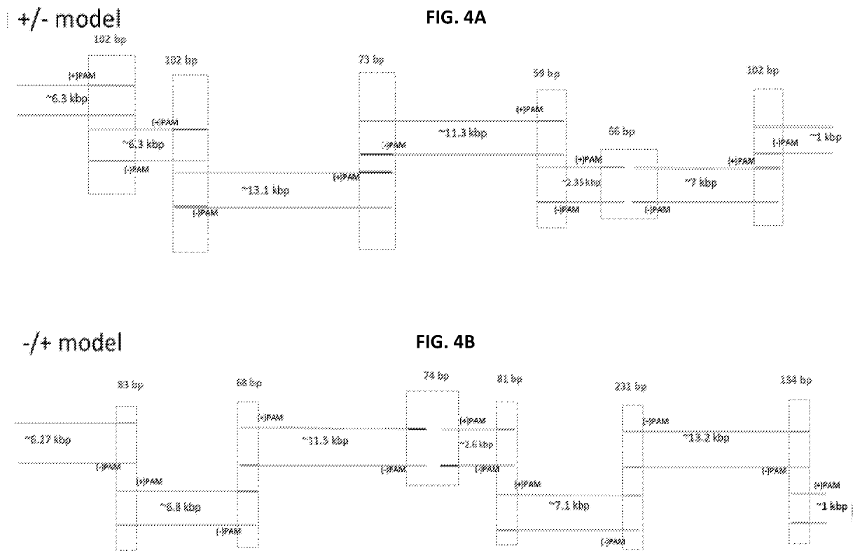

For each configuration, 6 pairs of sgRNA to fragment Lambda DNA were generated. The expected fragment as well as the linker sequence sizes are indicated as shown in FIG. 4A for the (+/−) sgRNA library and in FIG. 4B for the (−/+) sgRNA library.

Result 1: (+/−) and (−/+) with D10A Cas9 and H840A Cas9 with Denaturing or Taq Extension

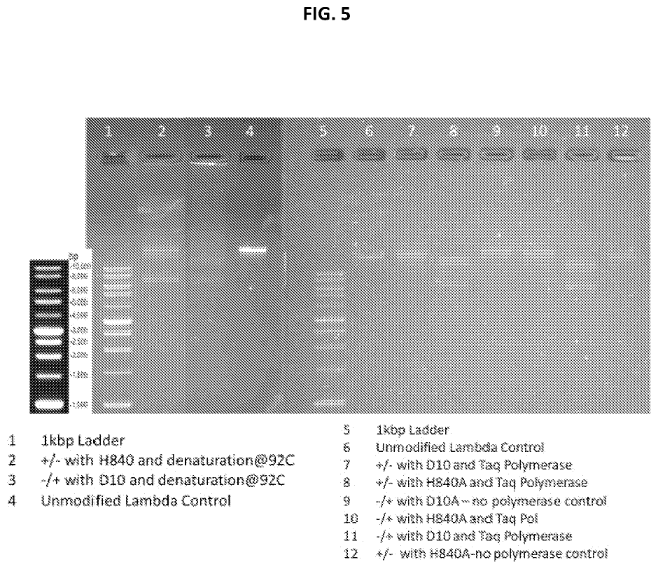

Lambda DNA was nicked with either D10A Cas9 or H840A Cas9 coupled with either (+/−) and (−/+) sgRNA for both enzymes. Following the nicking reaction, the DNA was either denatured or extended with Taq Polymerase. All samples were assessed with agarose gel electrophoresis. Results are shown in FIG. 5. Bands in Lanes 2 and 3 demonstrate successful nicking reaction. Bands in lanes 8 & 11 demonstrate successful DNA fragmentation in the (+/−) with H840A and (−/+) with D10A reactions. As expected, no fragmentation occurs in (+/−) with D10A (Lane 7) or with (−/+) with H840A (Lane10) reactions. Unmodified Lambda DNA (Lane 4, 6) and No Polymerase Temperature Control (Lanes 9, 12) are included as controls.

Result 2: (−/+) with D10A Cas9 and Extension with Vent Exo- or Klenow Exo-

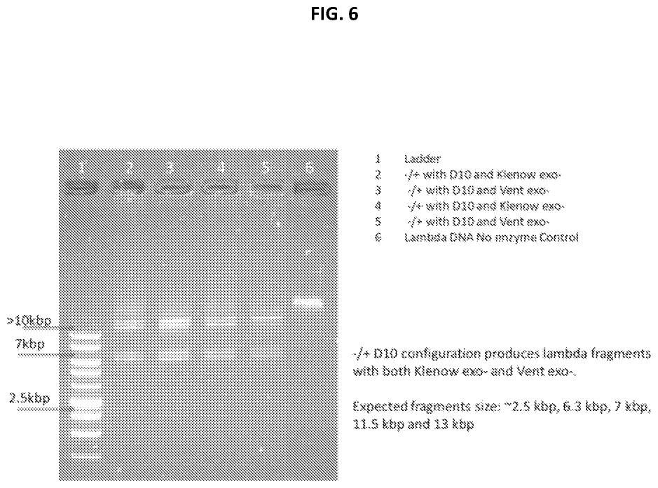

To prepare a sequencing library, a nicking reaction was performed on Lambda DNA using (−/+) sgRNA coupled with D10A Cas9. Following the nicking reaction, the DNA was extended with either Klenow exo- or Vent exo- polymerase. All samples were assessed with agarose gel electrophoresis. Results are shown in FIG. 6. Lanes 2 and 3 are samples from reactions with 300 ng Lambda DNA input and Lanes 4 & 5 are samples from reactions with 600 ng Lambda DNA input. Four or more bands are seen in in each lane indicating successful fragmentation. Lambda DNA with no enzymes is included as a control (Lane 6). Remaining sample from these reactions was used to prepare a nanopore sequencing library as described in Example 3.

### Example 3: Nanopore Sequencing

To demonstrate the presence of common shared sequence between adjacent fragments of the fragmented Lambda DNA, a sequencing library was prepared with the (−/+) D10A reaction from Example 2 and sequenced it with a Minion flowcell (Oxford Nanopore).

To prepare the sequencing library, 2.4 ug of fragmented DNA from the Linked fragmentation reactions was purified using Fragselect-I magnetic beads (AxyPrep) using beads to DNA ratio and quantified. The yield at this step was 35-45%.

Then, repair and end prep were performed on the purified DNA using NEBNext FFPE DNA Repair Mix, NEB M6630 and NEBNext Ultra II End-Repair/dA-tailing Module. In a 0.2 ml PCR tube, 47 uL of DNA sample (800 ng), 3.5 uL of FFPE Repair buffer, 2 uL Repair Mix, 3.5 uL of end prep reaction buffer and 3 uL of end prep enzyme mix were added. 1 uL of DNA Control Sequence (DNA CS) from the sequence ligation kit (SQK-LSK109, ONT) was also added as a positive control for this step. The mixture was incubated at 20° C. for 5 min and then at 65° C. for 5 min.

Next, the mixture was suspended in 62 μl of magnetic beads, incubated at room temperature on a rotator mixer for 5 min, washed twice with 200 μl fresh 70% ethanol, pellet allowed to dry for 2 min, and DNA eluted in 61 μl Nuclease Free Water. A 1 μl aliquot was quantified using Qubit Fluorometer.

Adapter Ligation was then performed by adding 5 μl Adaptor Mix and 25 uL of Ligation Buffer (SQK-LSK109 Ligation Sequencing Kit 1D, Oxford Nanopore Technologies (ONT)) and 10 μl NEBNextQuick T4 DNA Ligase to the 60 μl dA-tailed DNA, mixing gently and incubating at room temperature for 10 min.

The adaptor-ligated DNA was then cleaned up by adding a 40 μl of magnetic beads, incubating for 5 min at room temperature on a rotator mixer and resuspending the pellet in 250 μl Long Fragment Buffer (SQK-LSK109). The Purified mix was again incubated for 5 min at room temperature on the mixer and the pellet was resuspended in 15 uL of Elution Buffer (SQK-LSK109).

After incubating at room temperature for 10 min and pelleting the beads again, the supernatant (DNA library) was transferred to a new tube. A 1 μl aliquot was quantified using Qubit Fluorometer.

The loading mix was prepared immediately before use by adding 37.5 uL of Sequencing Buffer (SQK-LSK109) and 25.5 uL of Loading beads (SQK-LSK109) to 12 uL of DNA library.

SpotON flow cell was thawed and primed as instructed by the manufacturer before loading the library and starting the run. MinION sequencing was performed as per manufacturer's guidelines using FLO-MIN106 flongle flow cells from ONT. MinION sequencing was controlled using Oxford Nanopore Technologies MinKNOW software. Fast5 files were generated after completion of the reads. These Fast5 files were combined and converted to FASTQ for alignment. Integrated Genomics Viewer(igv) was used to align, filter & clean up the nanopore reads.

Results

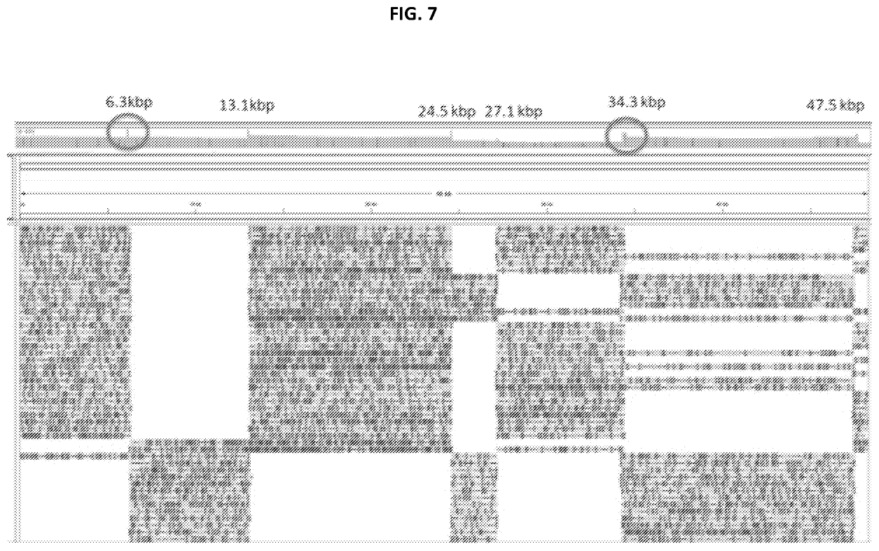

FIG. 7 shows reads aligned to the Lambda DNA reference. At the 6 expected fragmentation sites along the genome, an increase in coverage was observe. As predicted in the model, the use of six sgRNA in (−/+) configuration generated a total of 7 fragments. This is confirmed by FIG. 7. All nanopore reads are divided and arranged into 7 groups of the expected size fragments, i.e., 1 kbp, 2.5 kbp, 6.3 kbp, 6.8 kbp, 11.5 kbp, and 13 kbp.

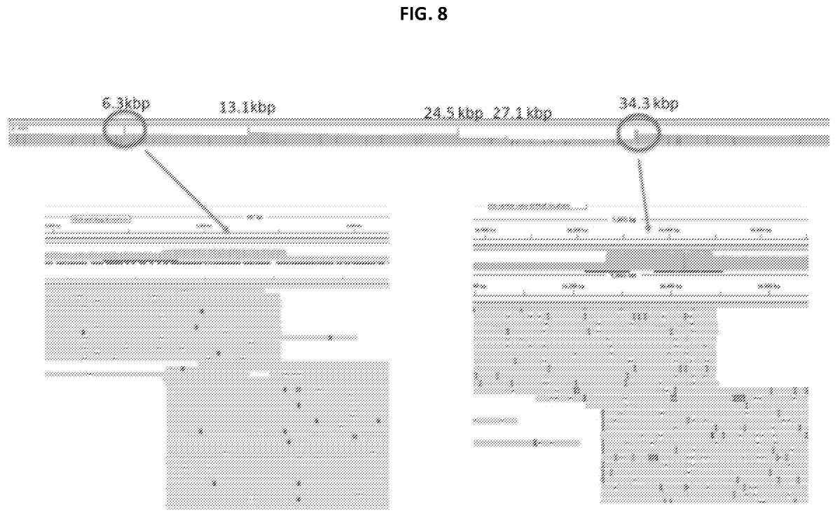

FIG. 8 presents a magnified view into two fragmentation sites, at 6.2 kbp and 34.4 kbp. A spike in coverage is seen at the ends of each read group. There is also an overlap observed between reads to the left and reads to the right. Together, the spike in coverage confirms the presence of an identical sequence at both fragment ends. The spiked coverage's beginning and end correspond to the extents of the shared segment between the adjacent fragments that were termed as linker sequence.

Each fragmentation site was set up to occur in between the (−/+) PAM pair on the dsDNA. For example, the first PAM site occurs around 6.27 kbp on the negative strand and the second PAM site occurs around 6.35 kbp on the positive strand.

Cas9 D10A-sgRNA complex nicks the opposite strand 3 bases away from each PAM site i.e., at 6272 on the positive strand and at 6355 on the negative strand. Therefore, the expected length of the first fragment is approximately 6.35 kbp. The shared linking sequence between this and the adjacent fragment is expected to be 83 bp long, which is the distance between both the nick sites. Then the read length obtained from the nanopore sequencing data corresponds to the fragment length with linker segments on one or both ends.

The linker segments varied lengths from about 60 bp to about 230 bp. The fragment lengths varied between 1000 bp to 13315 bp. This data is summarized by fragment number in Table 5. Further, the predicted lengths of the linker segments to the right of each fragment were compared with the lengths of the shared sequences on adjacent fragments obtained via nanopore sequencing data. In each fragment, the linker sequences mismatch by 1-2 bp but agree with each other. Further, each read length is also within 2 bp of the predicted fragment lengths. The differences in linker lengths are probably mainly due to differences in conventions in representing the nick locations in present predictions.

The read lengths obtained from sequencing data also second the bands obtained in gel electrophoresis in FIG. 6, i.e., 2.5 kbp, 6.3 kbp, 6.8 kbp, 11.5 kbp, and 13 kbp. The 1 kbp fragment was missing in the gel image but was present in the sequencing data.

TABLE 5
Comparison of Predicted Linker Segment &
Fragment Lengths to Shared sequence and average
read lengths from Nanopore Sequencing Data

| | | | Distance | |
|---|---|---|---|---|
| | | | between | |
| | | | Nick Sites | |
| | | | (Linker | |
| | | | Segment | Fragment |
| | Nick | Nick | Length on | Length |
| Predicted | Site 1 | Site 2 | the right end) | (bp) |
| End | 0 | | | 6355 |
| Fragment 1 | 6272 | 6355 | 83 | 6888 |
| Fragment 2 | 13092 | 13160 | 68 | 11553 |
| Fragment 3 | 24571 | 24645 | 74 | 2666 |
| Fragment 4 | 27156 | 27237 | 81 | 7338 |
| Fragment 5 | 34263 | 34494 | 231 | 13319 |
| Fragment 6 | 47448 | 47582 | 134 | 1054 |
| Fragment 7 | | 48502 | | |
| | | | Shared | |
| | | | Sequence | |
| | Spiked | Spiked | Length (bp) | Read |
| Nanopore | Coverage | Coverage | on the | Length |
| Reads | Start (bp) | End (bp) | right end | (bp) |
| End | 0 | | | 6355 |
| Fragment 1 | 6273 | 6355 | 82 | 6887 |
| Fragment 2 | 13093 | 13160 | 67 | 11552 |
| Fragment 3 | 24572 | 24645 | 73 | 2665 |
| Fragment 4 | 27157 | 27237 | 80 | 7338 |
| Fragment 5 | 34266 | 34495 | 229 | 13315 |
| Fragment 6 | 47449 | 47581 | 132 | 1053 |
| Fragment 7 | | 48502 | | |

Further, a comparison of the predicted linker lengths with the measured linker lengths from the sequencing data is shown in Table 6.

TABLE 6
Predicted and Measured Linker Lengths
Lambda DNA with cas9-D10A

| Predicted linker | Measured linker |
|---|---|
| length (bp) | length (bp) |
| 995 | 990 ± 50 |
| 57 | 67 ± 29 |
| 63 | 60 ± 16 |
| 785 | 787 ± 45 |
| 217 | 217 ± 15 |
| 123 | 123 ± 7 |

Lambda DNA with cas9-H840A

| Predicted linker | Measured linker |
|---|---|
| length (bp) | length (bp) |
| 213 | 195 ± 20 |
| 119 | 110 ± 21 |
| 90 | 87 ± 11 |
| 594 | 590 ± 32 |
| 444 | 440 ± 18 |
| 137 | 135 ± 22 |

To further examine the data, the complete sequences of predicted linker segments and the shared segments from nanopore reads on the Left (L) and the Right (R) ends of each fragment were compared. On comparison, it was observed that they were mismatched by 1-2 bp in each case and that the mismatches mainly occurs at the start or end of the sequences for each fragment.

Finally, was conclude that the data presented herein supports the proposed linked sequencing library model.

### Example 4: Long-Range PCR after 2-Step Ligation

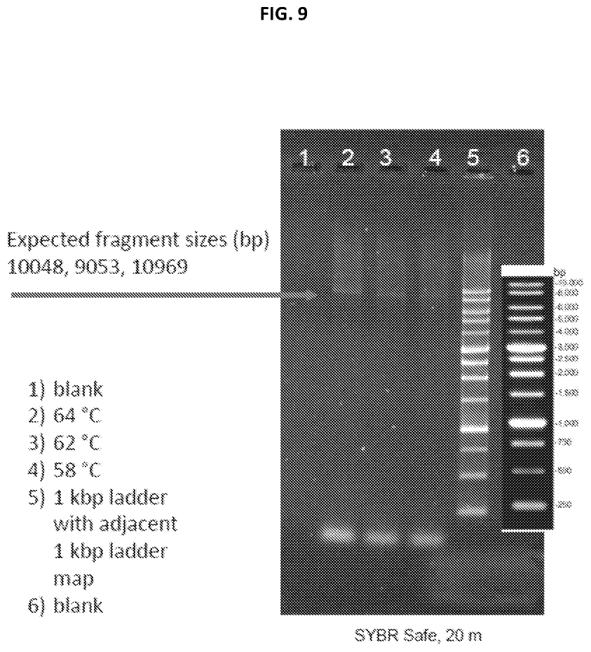

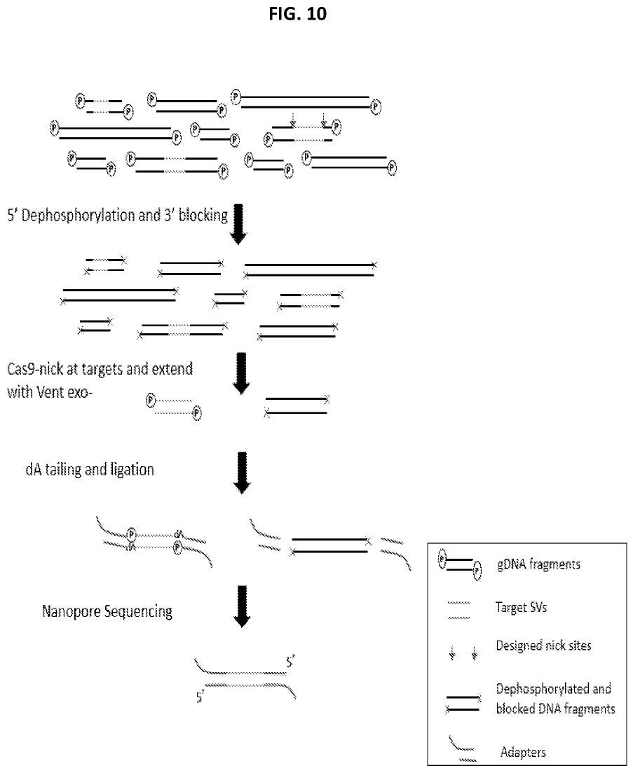

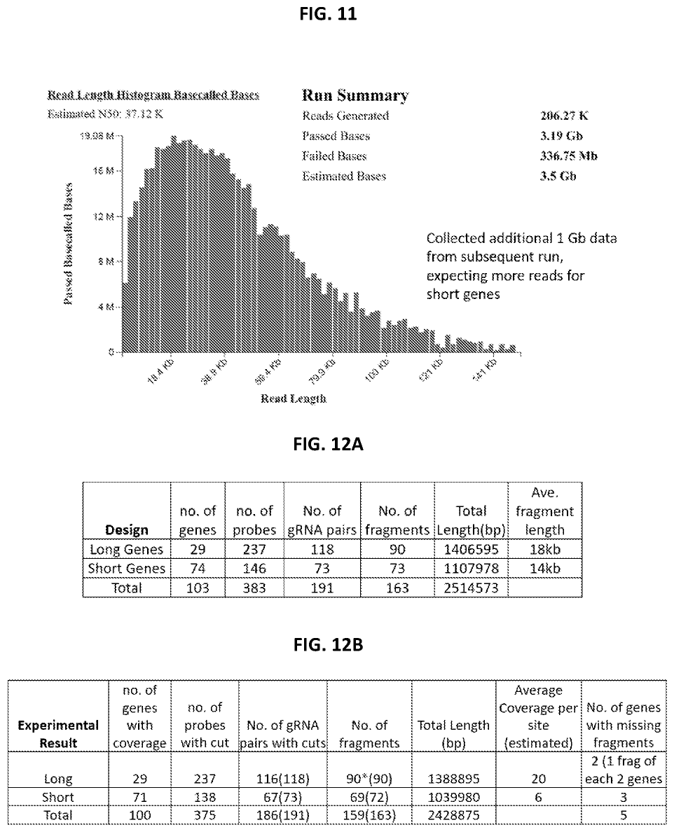

First, long DNA molecules were cut with Cas9-sgRNA nickase complex formed with multiple pairs of sgRNA. Each cut generated two complementary sticky ends. Second, after purification, the ligation adaptors that are complementary to half of the sticky ends were added and ligated to the end of DNA molecules. Third, after purification, the other half of the sticky ends were ligated with the rest of the adaptors. Finally, after purification, long-range PCR was performed with a pair of universal primers to amplify multiple long DNA fragments (10-20 kb). FIG. 9 shows a gel of the PCR amplified fragments after 2-step ligation of adapters.

### Example 5: Linked DNA Fragmentation and Nanopore Sequencing of H. influenzae Genomic DNA

Genomic DNA from the bacterium H. influenzae was fragmented using D10A Cas9-sgRNA complexes by the method described above for Lambda DNA. Nanopore sequencing of the generated linked-paired-end DNA fragments was performed as described above for Lambda DNA. A comparison of the predicted linker lengths with the measured linker lengths from the sequencing data is shown in Table 7.

TABLE 7
Predicted and Measured Linker Lengths
Hflu DNA with cas9-D10A

| Predicted linker | Measured linker |
|---|---|
| length (bp) | length (bp) |
| 476 | 471 ± 29 |
| 473 | 470 ± 23 |
| 439 | 417 ± 10 |
| 436 | 426 ± 19 |
| 417 | 411 ± 34 |
| 411 | 398 ± 21 |
| 404 | 400 ± 14 |
| 397 | 391 ± 12 |
| 394 | 386 ± 21 |
| 393 | 391 ± 6 |
| 389 | 381 ± 12 |
| 384 | 389 ± 21 |
| 378 | 368 ± 8 |
| 346 | 334 ± 12 |
| 344 | 341 ± 14 |
| 344 | 32 ± 14 |
| 330 | 321 ± 9 |
| 322 | 320 ± 12 |
| 311 | 311 ± 6 |
| 280 | 281 ± 2 |
| 278 | 268 ± 16 |
| 260 | 245 ± 9 |
| 232 | 222 ± 11 |
| 222 | 221 ± 8 |
| 215 | 211 ± 14 |
| 206 | 201 ± 17 |
| 194 | 184 ± 8 |
| 192 | 191 ± 7 |

### Example 6: Sequencing Human Genes

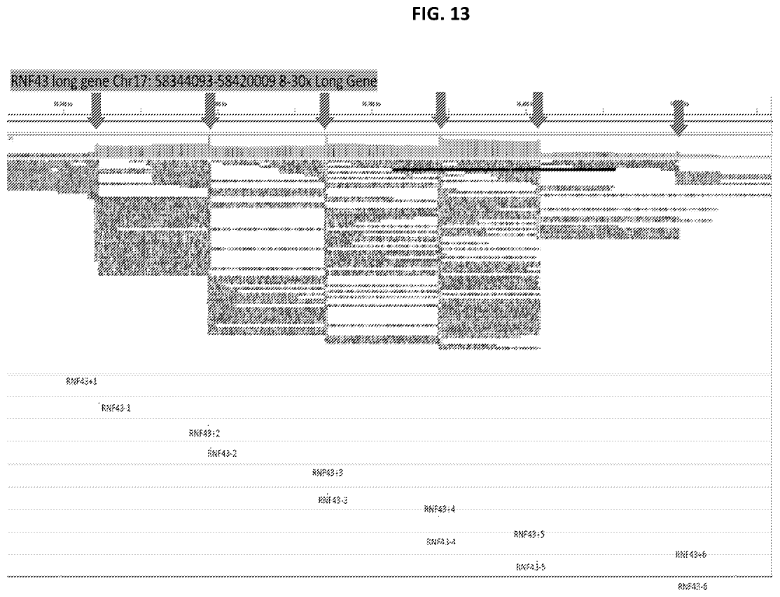

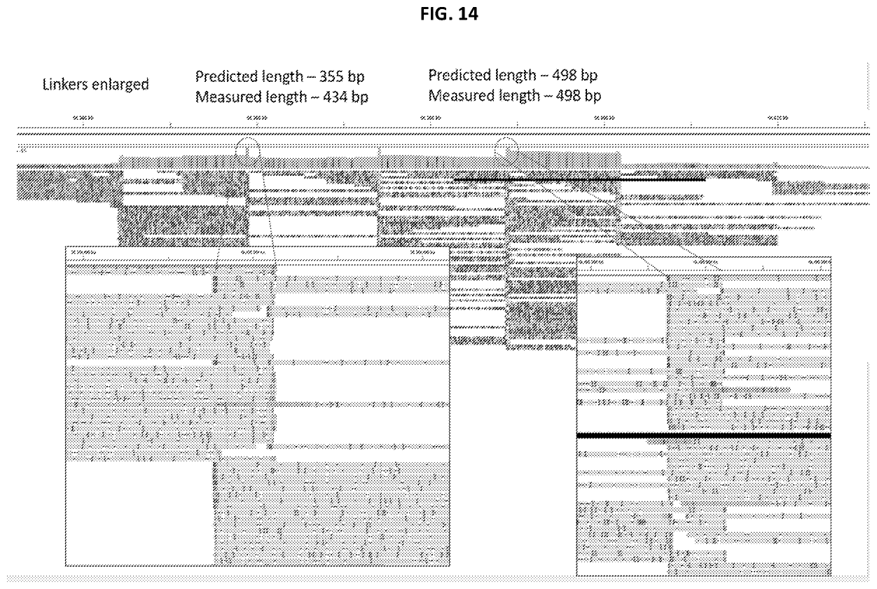

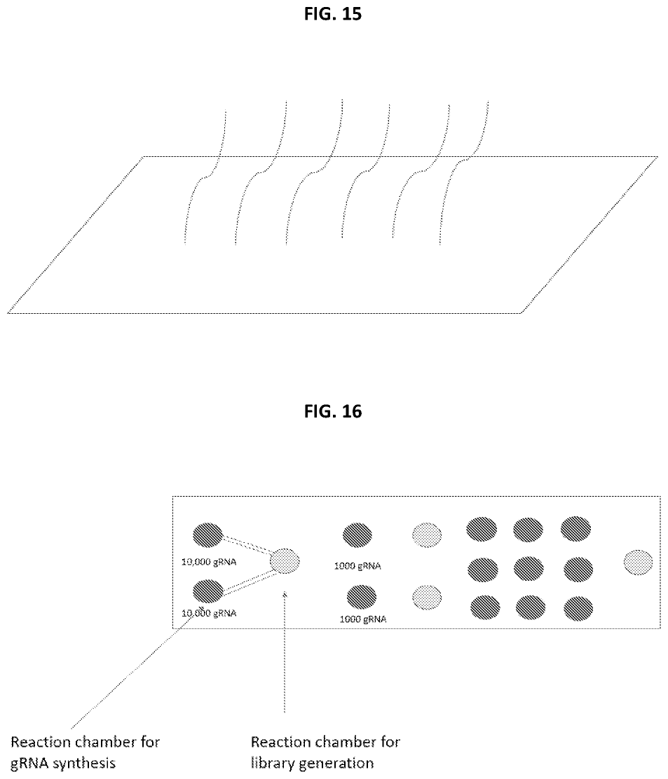

The methods of the invention were further tested to sequence human genes. For this, an sgRNA library for sequencing 103 human genes was constructed. Details of the sgRNA library are presented in FIG. 12A. Out of 103 human genes, 100 genes were successfully sequenced, and the results are presented in FIG. 12B. As an example, FIGS. 13 and 14 show the nanopore reads for RNF43 gene, which is one of the 100 genes that was sequenced.

Summary of the Methods of the Invention: Generation and Sequencing of Linked-Paired-End Fragments and their Advantages Over Current Technologies.

As described previously herein, the methods of the present invention include methods of fragmenting a double-stranded DNA sample such as a whole genome so that the ends of the adjacent DNA fragments share common linker sequences. These linker sequences are normally about 50 bases long or more, such as about 50 to about 1000 bp.

The linked DNA fragments are either circularized to form linked-paired-end sequencing library, and/or directly shotgun sequenced. In the case of the linked-paired-end sequencing library, additional 100-200 bases on both sides of the linker sequences (paired-end sequences), along with the linker sequences, are read with next generation sequencing technology (FIG. 7 and FIG. 8). This sequencing information is used to construct a de novo whole genome map as exemplified herein for bacteriophage lambda genome. This method will capture various scales of contiguity information at a throughput commensurate with the current scale of massively parallel sequencing, and extend the use of the short read sequencing technology in de novo genome assembly, structural variation detection, and haplotype-resolved genome sequencing. In the case of shotgun sequencing, the linked DNA fragments are shotgun sequenced by dilution, amplification, and the sequence reads can then be mapped back to the whole genome map, assembled with linked-paired-end sequencing library.

The linked-paired-end sequencing methods of the present invention offer a unique, high-throughput approach to address the main issues of short-read sequencing technology without introducing any additional equipment.

Based on linked-paired-end sequencing methods, the haplotype-scaffold sequencing (HSS) generates a haplotype-resolved scaffold, whose contiguity matches with shotgun, short reads contig size. This allows direct use for supporting de novo assembly of complex genomes. The HSS procedure can be easily integrated into standard sequencing protocol (e.g. Illumina sequencing). Since the methods of the invention relate only to sequencing a small portion of the genome, they do not add any significant cost to whole genome shotgun sequencing. The linked-paired-end sequencing libraries of the present invention can be run together with other shotgun sequencing libraries.

The methods of this invention rely on sequencing the DNA fragments generated at certain sequence motifs and provides more structured sequence contiguity than traditional mate-pair library, which relies on randomly sheared fragments and requires more coverage to provide full linkage. The procedures provided herein are much simpler than the stochastic separation of sequencing fragments, as they do not require thousands of pools and sequencing barcodes. Based on linked-paired-end libraries, the HSS generates internal barcodes (from about 50 to about 1000 bp) between the sequencing fragments and thus provides higher resolution and more information content than classical genome mapping. Because the methods of the invention provide up to about 1000 bp at sequence motif sites, instead of only a few bases as is the case in the conventional genome mapping, denser nicking sites within the genome, limited only by the number and relative locations of PAM sequences, can be used because they will not be limited by optical resolution. Additionally, only about 10× sequencing coverage is sufficient to achieve a good result.

In summary by using the methods of the present invention, high-quality, low-cost de novo assembly of complex genomes is made possible.

### Enumerated Embodiments

The following exemplary embodiments are provided, the numbering of which is not to be construed as designating levels of importance:

Embodiment 1 provides a method of preparing a DNA sequencing library comprising DNA fragments having linked-paired ends from at least one double-stranded DNA sample having a first and a second DNA strand, the method comprising:
a. obtaining a single guide RNA (sgRNA) library comprising multiple sgRNA pairs, wherein:
i. each sgRNA pair comprises a first sgRNA and a second sgRNA, and
ii. the first sgRNA of each sgRNA pair targets a first target DNA sequence on the first DNA strand and the second sgRNA of each sgRNA pair targets a second target DNA sequence on the second DNA strand;
b. contacting the double-stranded DNA sample with the sgRNA library and at least one nickase, wherein the nickase comprises at least one RNA-guided endonuclease having a single active endonuclease domain, thereby forming a nick within each first and each second target DNA sequence; and
c. contacting the double-stranded DNA sample with a strand-displacing polymerase and one or more nucleotides, thereby forming a single-stranded flap on the double-stranded DNA sample beginning at each nick of step (b), wherein each single-stranded flap hybridizes to its corresponding complementary strand of the double stranded DNA sample, thereby generating linked-paired-end DNA fragments.

Embodiment 2 provides the method of Embodiment 1, wherein the first target DNA sequence and the second target DNA sequence of each sgRNA pair is located adjacent to a protospacer adjacent motif (PAM) sequence.

Embodiment 3 provides a method of preparing a DNA sequencing library comprising DNA fragments having linked-paired ends from at least one double-stranded DNA sample having a first and a second DNA strand, the method comprising:
a. obtaining a single guide RNA (sgRNA) library comprising multiple sgRNAs, wherein each sgRNA targets a first target DNA sequence on the first DNA strand;
b. contacting the double-stranded DNA sample with the sgRNA library and at least one first nickase, wherein the first nickase comprises at least one RNA-guided endonuclease having a single active endonuclease domain, thereby forming a nick within each first target DNA sequence;
c. contacting the double-stranded DNA sample with at least one second nickase, wherein the second nickase comprises a nicking restriction endonuclease which targets a second target DNA sequence on the second DNA strand, thereby forming a nick within each second target DNA sequence, wherein step (b) and step (c) may be performed in any order or simultaneously; and
d. contacting the double-stranded DNA sample with a strand-displacing polymerase and one or more nucleotides, thereby forming a single-stranded flap on the double-stranded DNA sample beginning at each nick of steps (b) and (c), wherein each single-stranded flap hybridizes to its corresponding complementary strand of the double stranded DNA sample, thereby generating linked-paired-end DNA fragments.

Embodiment 4 provides a method of Embodiment 3, wherein the first target DNA sequence of each sgRNA is located adjacent to a protospacer adjacent motif (PAM) sequence.

Embodiment 5 provides the method of Embodiment 3 or 4, wherein the nicking restriction endonuclease comprises one or more endonucleases selected from the group consisting of Nb.BbvCI, Nt.BbvCI, Nt.Bsml, Nt.BsmAI, Nt.BstNBI, Nb.BsrDI, Nb.BstI, Nt.BspQI, Nt.BpulOI and Nt.Bpul0I.

Embodiment 6 provides the method of any one of the preceding Embodiments, further comprising inactivating the nickase(s).

Embodiment 7 provides a method of any one of the preceding Embodiments, wherein the sgRNA library is computationally designed to target sequences within the double-stranded DNA sample.

Embodiment 8 provides the method of any one of the preceding Embodiments, wherein the first target DNA sequence and the second target DNA sequence are separated by about 50 to about 1000 base pairs (bp) of the double-stranded DNA sample.

Embodiment 9 provides the method of any one of the preceding Embodiments, wherein each linked-paired-end DNA fragment comprises a linker sequence at each end of the DNA fragment, wherein each linker sequence comprises from about 50 to about 1000 bp of DNA sequence which is at least 90%, at least 95%, at least 98%, at least 99%, or at least 100% identical to a linker sequence of an adjacent DNA fragment.

Embodiment 10 provides the method of any one of the preceding Embodiments, wherein the sgRNA library comprises at least 5, at least 10, at least 25, at least 50, at least 100, at least 250, at least 500, at least 600, at least 700, at least 800, at least 900, or at least 1000 distinct sgRNAs.

Embodiment 11 provides the method of any one of the preceding Embodiments, wherein obtaining the sgRNA library comprises synthesizing the sgRNA library in a single reaction.

Embodiment 12 provides the method of Embodiment 11, wherein synthesizing the multiple sgRNAs in a single reaction comprises:
i. obtaining a dsDNA duplex library wherein each dsDNA duplex comprises a T7 promoter sequence operably linked to a sequence encoding an sgRNA, and further wherein the dsDNA duplex library is treated with exonuclease, preferably at about 37° C. for about 1 hour, and purified to remove single-stranded DNA (ssDNA);
ii. contacting the dsDNA duplex library of step (i) with T7 RNA polymerase and NTPs, preferably at about 37° C. for about 2 hours, thereby synthesizing the sgRNA library;
iii. contacting the dsDNA duplex library of step (ii) with DNase I, preferably at about 37° C. for about 15 minutes, thereby degrading the dsDNA duplexes; and
iv. optionally purifying and/or quantifying the sgRNA library.

Embodiment 13 provides the method of any one of the preceding Embodiments, wherein the RNA-guided endonuclease is a clustered regularly interspaced short palindromic repeat (CRISPR)-associated endonuclease selected from a Cas9 and a Cas12a (Cpf1).

Embodiment 14 provides the method of any one of the preceding Embodiments, wherein the RNA-guided endonuclease is D10A Cas9 or H840A Cas9.

Embodiment 15 provides the method any one of the preceding Embodiments, wherein the strand-displacing polymerase comprises Klenow Fragment or D141A/E143A Thermococcus litoralis (“Vent exo-”) DNA polymerase.

Embodiment 16 provides the method of any one of the preceding Embodiments, wherein the linked-paired-end DNA fragments range in size from about 100 bp up to about 1,000,000 bp (1 Mbp) or more.

Embodiment 17 provides the method of any one of the preceding Embodiments, wherein the linked-paired-end DNA fragments range in size from about 100 bp up to about 20,000 bp.

Embodiment 18 provides the method of any one of the preceding Embodiments, wherein the linked-paired-end DNA fragments are uniformly spaced within the double-stranded DNA sample.

Embodiment 19 provides the method of any one of the preceding Embodiments, wherein the double-stranded DNA sample comprises at least one genome selected from a viral genome, a bacterial genome, an archaeal genome, a fungal genome, a plant genome, an animal genome, a mammalian genome, and a human genome.

Embodiment 20 provides the method of any one of the preceding Embodiments, wherein the double-stranded DNA sample comprises a mixture of genomes, wherein the mixture of genomes comprises at least two genomes and up to about 10, about 50, about 100, about 500, about 1000, about 2000, or about 3000 or more genomes.

Embodiment 21 provides the method of any one of the preceding Embodiments, further comprising modifying the generated linked-paired-end DNA fragments with repair enzymes, 3′-deoxyadenosine (dA) tail addition, and/or adapter ligation.

Embodiment 22 provides the method of any one of the preceding Embodiments, wherein the generated linked-paired-end DNA fragments are further processed such that each linked-paired-end DNA fragment is 5′-phosphorylated and comprises a 3′-dA tail.

Embodiment 23 provides the method of any one of the preceding Embodiments, further comprising (a) circularizing the linked-paired-end fragments, (b) fragmenting the circularized fragments, (c) size selecting the fragments of interest from step (b), and ligating adapters to the fragments of interest.

Embodiment 24 provides the method of any one of the preceding Embodiments, wherein each of the generated linked-paired-end DNA fragments is ligated to a pair of universal adapters and amplified by long-range PCR.

Embodiment 25 provides the method of any one of the preceding Embodiments, further comprising sequencing the generated linked-paired-end DNA fragments with a high throughput sequencing platform.

Embodiment 26 provides the method of Embodiment 25, wherein the high throughput sequencing platform is selected from the group consisting of Illumina sequencing, SOLiD sequencing, 454 pyrosequencing, Ion Torrent semiconductor sequencing, single molecule real-time (SMRT) circular consensus sequencing, and nanopore (MinION) sequencing.

Embodiment 27 provides the method of Embodiment 26, wherein the high throughput sequencing platform is nanopore (MinION) sequencing.

Embodiment 28 provides a method of generating at least one de novo whole genome map, the method comprising:
a. sequencing the DNA sequencing library prepared by the method of any one of the preceding claims with a high throughput sequencing platform, thereby generating sequence reads; and
b. computationally processing the sequence reads to align adjacent linker sequences, thereby ordering the linked-paired-end DNA fragments and generating the at least one de novo whole genome map.

Embodiment 29 provides the method of Embodiment 28, wherein the sequencing comprises at least 10× sequencing coverage.

Embodiment 30 provides the method of Embodiment 28 or 29, wherein computationally processing the sequence reads further comprises correlating the sequence reads to a sequence assembly, a genetic or cytogenetic map, a structural pattern, a structural variation including insertions and deletions, a physiological characteristic, a methylation pattern, an epigenomic pattern, a location of a CpG island, a single nucleotide polymorphism (SNP), a copy number variation (CNV), or a combination thereof.

Embodiment 31 provides the method of any one of Embodiments 28 to 30, wherein the processing further comprises assembly of a haplotype sequence.

Embodiment 32 provides the method of Embodiment 31, wherein the haplotype sequence comprises a major histocompatibility (MEW) region of a mammalian genome, preferably a human genome.

Embodiment 33 provides the method Embodiment 28, wherein the method of generating the genome map comprises sequencing both introns and exons within a gene.

Embodiment 34 provides a microdevice for generating sgRNA library and a DNA sequencing library, wherein the device comprises
a. a first substrate having a first surface; and
b. a plurality of recessed portions from the first surface into the first substrate, wherein each of the plurality of the recessed portions comprises either a microwell or a micro flow channel;
wherein each of the plurality of microwells is used for generating either the sgRNA library or for generating the DNA sequencing library, and
wherein each of the plurality of microwells used for generating the sgRNA library is in fluidic communication with at least one microwell used for generating the DNA sequencing library.

Embodiment 35 provides a method of generating sgRNA on a surface of a substrate,
wherein the method comprises generating sgRNA library using single stranded (ss) oligonucleotides; and
wherein the ss oligonucleotides are synthesized directly on the surface using photolithography.

Embodiment 36 provides a method of Embodiment 35, wherein about one million sgRNAs can be simultaneously generated on the surface.

Embodiment 37 provides a method of Embodiment 35, wherein the substrate is a glass.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of preparing a DNA sequencing library comprising DNA fragments having linked-paired ends from at least one double-stranded DNA sample having a first and a second DNA strand, the method comprising:
a. obtaining a single guide RNA (sgRNA) library comprising multiple sgRNA pairs, wherein:
i. each sgRNA pair comprises a first sgRNA and a second sgRNA, and
ii. the first sgRNA of each sgRNA pair targets a first target DNA sequence on the first DNA strand and the second sgRNA of each sgRNA pair targets a second target DNA sequence on the second DNA strand;
b. contacting the double-stranded DNA sample with the sgRNA library and at least one nickase, wherein the nickase comprises at least one RNA-guided endonuclease having a single active endonuclease domain, thereby forming a nick within each first and each second target DNA sequence; and
c. contacting the double-stranded DNA sample with a strand-displacing polymerase and one or more nucleotides, thereby forming a single-stranded flap on the double-stranded DNA sample beginning at each nick of step (b), wherein each single-stranded flap hybridizes to its corresponding complementary strand of the double stranded DNA sample, thereby generating linked-paired-end DNA fragments.

2. The method of claim 1, wherein the first target DNA sequence and the second target DNA sequence of each sgRNA pair is located adjacent to a protospacer adjacent motif (PAM) sequence.

3. A method of preparing a DNA sequencing library comprising DNA fragments having linked-paired ends from at least one double-stranded DNA sample having a first and a second DNA strand, the method comprising:
a. obtaining a single guide RNA (sgRNA) library comprising multiple sgRNAs, wherein each sgRNA targets a first target DNA sequence on the first DNA strand;
b. contacting the double-stranded DNA sample with the sgRNA library and at least one first nickase, wherein the first nickase comprises at least one RNA-guided endonuclease having a single active endonuclease domain, thereby forming a nick within each first target DNA sequence;
c. contacting the double-stranded DNA sample with at least one second nickase, wherein the second nickase comprises a nicking restriction endonuclease which targets a second target DNA sequence on the second DNA strand, thereby forming a nick within each second target DNA sequence, wherein step (b) and step (c) may be performed in any order or simultaneously; and
d. contacting the double-stranded DNA sample with a strand-displacing polymerase and one or more nucleotides, thereby forming a single-stranded flap on the double-stranded DNA sample beginning at each nick of steps (b) and (c), wherein each single-stranded flap hybridizes to its corresponding complementary strand of the double stranded DNA sample, thereby generating linked-paired-end DNA fragments.

4. The method of claim 3, wherein the first target DNA sequence of each sgRNA is located adjacent to a protospacer adjacent motif (PAM) sequence.

5. The method of claim 3, wherein the nicking restriction endonuclease comprises one or more endonucleases selected from the group consisting of Nb.BbvCI, Nt.BbvCI, Nt.Bsml, Nt.BsmAI, Nt.BstNBI, Nb.BsrDI, Nb.BstI, Nt.BspQI, Nt.BpulOI and Nt.Bpul0I.

6. The method of claim 1, further comprising inactivating the nickase(s).

7. The method of claim 1, wherein the sgRNA library is computationally designed to target sequences within the double-stranded DNA sample.

8. The method of claim 1, wherein the first target DNA sequence and the second target DNA sequence are separated by about 50 to about 1000 base pairs (bp) of the double-stranded DNA sample.

9. The method of claim 1, wherein each linked-paired-end DNA fragment comprises a linker sequence at each end of the DNA fragment, wherein each linker sequence comprises from about 50 to about 1000 bp of DNA sequence which is at least 90%, at least 95%, at least 98%, at least 99%, or at least 100% identical to a linker sequence of an adjacent DNA fragment.

10. The method of claim 1, wherein the sgRNA library comprises at least 5, at least 10, at least 25, at least 50, at least 100, at least 250, at least 500, at least 600, at least 700, at least 800, at least 900, or at least 1000 distinct sgRNAs.

11. The method of claim 1, wherein obtaining the sgRNA library comprises synthesizing the sgRNA library in a single reaction.

12. The method of claim 11, wherein synthesizing the multiple sgRNAs in a single reaction comprises:
i. obtaining a dsDNA duplex library wherein each dsDNA duplex comprises a T7 promoter sequence operably linked to a sequence encoding an sgRNA, and further wherein the dsDNA duplex library is treated with exonuclease, preferably at about 37° C. for about 1 hour, and purified to remove single-stranded DNA (ssDNA);
ii. contacting the dsDNA duplex library of step (i) with T7 RNA polymerase and NTPs, preferably at about 37° C. for about 2 hours, thereby synthesizing the sgRNA library;
iii. contacting the dsDNA duplex library of step (ii) with DNase I, preferably at about 37° C. for about 15 minutes, thereby degrading the dsDNA duplexes; and
iv. optionally purifying and/or quantifying the sgRNA library.

13. The method of claim 1, wherein the RNA-guided endonuclease is a clustered regularly interspaced short palindromic repeat (CRISPR)-associated endonuclease selected from a Cas9 and a Cas12a (Cpf1).

14. The method of claim 1, wherein the RNA-guided endonuclease is D10A Cas9 or H840A Cas9.

15. The method of claim 1, wherein the strand-displacing polymerase comprises Klenow Fragment or D141A/E143A Thermococcus litoralis (“Vent exo-”) DNA polymerase.

16. The method of claim 1, wherein the linked-paired-end DNA fragments range in size from about 100 bp up to about 1,000,000 bp (1 Mbp) or more.

17. The method of claim 1, wherein the linked-paired-end DNA fragments range in size from about 100 bp up to about 20,000 bp.

18. The method of claim 1, wherein the linked-paired-end DNA fragments are uniformly spaced within the double-stranded DNA sample.

19. The method of claim 1, wherein the double-stranded DNA sample comprises at least one genome selected from a viral genome, a bacterial genome, an archaeal genome, a fungal genome, a plant genome, an animal genome, a mammalian genome, and a human genome.

20. The method of claim 1, wherein the double-stranded DNA sample comprises a mixture of genomes, wherein the mixture of genomes comprises at least two genomes and up to about 10, about 50, about 100, about 500, about 1000, about 2000, or about 3000 or more genomes.

21. The method of claim 1, further comprising modifying the generated linked-paired-end DNA fragments with repair enzymes, 3′-deoxyadenosine (dA) tail addition, and/or adapter ligation.

22. The method of claim 1, wherein the generated linked-paired-end DNA fragments are further processed such that each linked-paired-end DNA fragment is 5′-phosphorylated and comprises a 3′-dA tail.

23. The method of claim 1, further comprising (a) circularizing the linked-paired-end fragments, (b) fragmenting the circularized fragments, (c) size selecting the fragments of interest from step (b), and ligating adapters to the fragments of interest.

24. The method of claim 1, wherein each of the generated linked-paired-end DNA fragments is ligated to a pair of universal adapters and amplified by long-range PCR.

25. The method of claim 1, further comprising sequencing the generated linked-paired-end DNA fragments with a high throughput sequencing platform.

26. The method of claim 25, wherein the high throughput sequencing platform is selected from the group consisting of Illumina sequencing, SOLiD sequencing, 454 pyrosequencing, Ion Torrent semiconductor sequencing, single molecule real-time (SMRT) circular consensus sequencing, and nanopore (MinION) sequencing.

27. The method of claim 26, wherein the high throughput sequencing platform is nanopore (MinION) sequencing.

28. A method of generating at least one de novo whole genome map, the method comprising:
a. sequencing the DNA sequencing library prepared by the method of claim 1 with a high throughput sequencing platform, thereby generating sequence reads; and
b. computationally processing the sequence reads to align adjacent linker sequences, thereby ordering the linked-paired-end DNA fragments and generating the at least one de novo whole genome map.

29. The method of claim 28, wherein the sequencing comprises at least 10× sequencing coverage.

30. The method of claim 28, wherein computationally processing the sequence reads further comprises correlating the sequence reads to a sequence assembly, a genetic or cytogenetic map, a structural pattern, a structural variation, a physiological characteristic, a methylation pattern, an epigenomic pattern, a location of a CpG island, a single nucleotide polymorphism (SNP), a copy number variation (CNV), or a combination thereof.

31. The method of claim 28, wherein the processing further comprises assembly of a haplotype sequence.

32. The method of claim 31, wherein the haplotype sequence comprises a major histocompatibility (WIC) region of a mammalian genome, preferably a human genome.

33. The method of claim 28, wherein the method of generating genome maps comprises sequencing entire gene including its introns and exons.

34. A microdevice for generating sgRNA library and a DNA sequencing library, wherein the device comprises
a. a first substrate having a first surface; and
b. a plurality of recessed portions extending from the first surface into the first substrate, wherein each of the plurality of the recessed portions comprises either a microwell or a micro flow channel;
wherein each of the plurality of microwells is used for generating either the sgRNA library or for generating the DNA sequencing library, and
wherein each of the plurality of microwells used for generating the sgRNA library is in fluidic communication with at least one microwell used for generating the DNA sequencing library.

35. A method of generating sgRNA on a surface of a substrate,
wherein the method comprises generating sgRNA library using single-stranded (ss) oligonucleotides; and
wherein the ss oligonucleotides are synthesized directly on the surface using photolithography.

36. The method of claim 35, wherein about one million sgRNAs can be simultaneously generated on the surface.

37. The method of claim 35, wherein the substrate is a glass.
